(19) United States
(12) Patent Application Publication
(10) Pub. No.: US 2024/0180690 A1
(43) Pub. Date: Jun. 6, 2024

(54) SPECTACLE-FREE ACCOMMODATING LENS (SFAL)

(71) Applicant: Khalid Mentak, San Ramon, CA (US)

(72) Inventor: Khalid Mentak, San Ramon, CA (US)

(21) Appl. No.: 16/612,116

(22) PCT Filed: Jul. 6, 2018
(86) PCT No.: PCT/US18/31867

§ 371 Date: Nov. 8, 2019

Related U.S. Application Data
(60) Provisional application No. 62/503,691, filed on May 9, 2017.

(51) Int. Cl. A61F 2/16

(52) U.S. Cl. CPC A61F 2/1635; A61F 2/1648; A61F 2002/1682; A61F 2220/0091; A61F 2250/0003

(57) ABSTRACT

The disclosure relates to intraocular lenses. More particularly, the disclosure relates to intraocular lenses that alter the refractive power of the eye in response to changes in the tension of the ciliary muscle or ciliary body of the eye. Lenses disclosed herein are referred to as accommodating lenses.

### CROSS REFERENCE TO RELATED APPLICATIONS

This application claims priority to and is a non-provisional application of U.S. Patent Application No. 62/503,691 filed May 9, 2017, which is incorporated herein in its entirety.

### FIELD

The disclosure relates to intraocular lenses. More particularly, the disclosure relates to intraocular lenses that alter the refractive power of the eye in response to changes in the tension of the ciliary muscle or ciliary body of the eye. Lenses disclosed herein are referred to as accommodating lenses.

### BACKGROUND

The natural lens of a human eye is a transparent crystalline body, which is contained within a capsular bag located behind the iris and in front of the vitreous cavity in a region known as the posterior chamber. The capsular bag is attached on all sides by fibers, called zonules, to a muscular ciliary body. At its rear, the vitreous cavity, which is filled with a gel, further includes the retina, on which light rays passing through the lens are focused. Contraction and relaxation of the ciliary bodies changes the shape of the bag and of the natural lens therein, thereby enabling the eye to focus light rays on the retina originating from objects at various distances.

Cataracts occur when the natural lens of the eye or of its surrounding transparent membrane becomes clouded and obstructs the passage of light resulting in various degrees of blindness. To correct this condition in a patient, a surgical procedure is performed in which the clouded natural lens, or cataract, is extracted and replaced by an artificial intraocular lens. During cataract surgery, the anterior portion of the capsular bag is removed along with the cataract, and the posterior portion of the capsular bag, called the posterior capsule, is sometimes left intact to serve as a support site for implanting the intraocular lens. Such lenses, however, have the drawback that they have a fixed refractive power and are therefore unable to change their focus.

Various types of intraocular lens with the capability of altering their refractive power have been suggested in an effort to duplicate the performance of the natural lens within the eye. Such accommodating intraocular lenses, as they are known in the art, have a variety of designs directed to enable the patient to focus on, and thereby clearly see, objects located at a plurality of distances. Examples may be found in such publications as U.S. Pat. Nos. 4,254,509; 4,932,966; 6,299,641; and 6,406,494.

U.S. Pat. No. 5,443,506 to Garabet discloses a variable focus intraocular lens, which alters the medium between the two surfaces of the lens to alter its accommodation. The lens of the '506 patent has continuous flow loops, which couple a channel in first portion of the intraocular lens. The continuous flow loops, in addition to providing a channel, provide the means by which the intraocular lens is positioned and held in the eye. In some circumstances, the continuous flow loop(s) comprise the lens haptics.

U.S. Pat. No. 5,489,302 discloses an accommodating intraocular lens for implantation in the posterior chamber of the eye. This lens comprises a short tubular rigid frame and transparent and resilient membrane attached thereto at its bases. The frame and the membranes confine a sealed space filled with a gas. The frame includes flexible regions attached via haptics to the posterior capsule. Upon stretching of the capsule by the eye's ciliary muscles, the flexible regions are pulled apart, thereby increasing the volume and decreasing the pressure within the sealed space. This changes the curvature of the membranes and accordingly, the refractive power of the lens.

U.S. Pat. No. 6,117,171 discloses an accommodating intraocular lens that is contained inside an encapsulating rigid shell so as to make it substantially insensitive to changes in the intraocular environment. The lens is adapted to be implanted within the posterior capsule and comprises a flexible transparent membrane, which divides the interior of the intraocular lens into separate front and rear spaces, each filled with a fluid having a different refractive index. The periphery of the rear space is attached to haptics, which are in turn attached to the posterior capsule. Upon stretching of the capsule by the eye's ciliary muscles, the haptics and hence this periphery is twisted apart to increase the volume of rear space and changes the pressure difference between the spaces. As a result, the curvature of the membrane and accordingly, the refractive power of the lens changes.

Another approach to varying the focus of an IOL is to form a conventional hard intraocular lens with a flexible outer surface made from a material such as silicone. Water is then injected in between the conventional hard portion of the lens and the flexible outer surface of the lens. The water will stretch the outer flexible layer to change the radius of curvature of the intraocular lens and thereby change the accommodation of the lens. One disadvantage of this approach is that a fluid source, a fluid pump and a flow control valve all must be provided within close proximity to the lens. As the area around the crystalline lens of the eye is quite confined, most of the fluid injection components have to be provided on the lens itself. Further, an energy source must be provided to pump the fluid. As there is no mechanical force generated in the eye that is strong enough to pump the fluid, an external power supply is required to run the pump. Such an external power supply is usually implemented using a battery which has a limited life cycle.

A further approach that has been used to vary the accommodation of an IOL is the coating of a conventional IOL with a liquid crystal material. A voltage source is applied to the crystal material to polarize the crystals. Once the crystals are polarized the refractive index of the crystalline material changes thereby changing the accommodation of the IOL. One principal disadvantage of this type of system is the relatively large amount of energy that is required to polarize the liquid crystal material, on the order of 25 volts. As there is no known manner of generating that level of voltage within the body, an external power source, such as a battery, is therefore necessary.

Some conventional accommodating IOLs rely on hydraulic systems to cause various fluids to reshape the refractive surface of the IOL. Such AIOLs have severe limitations stemming from the complexity of moving fluids in a precise manner and the high risk of leaking fluids into ocular tissues. Most importantly, after removal of the natural lens, the capsular bag becomes somewhat opaque and less flexible due to fibrosis and posterior capsule opacification (PCO). This reduces the ability of the IOL to harvest the ciliary muscle forces necessary for the functioning of the AIOL. Other accommodating IOLs involve a displacement of the whole IOL along the optical axis to create accommodation. This does not only require a relatively larger force but also fails to deliver larger changes in diopter due to the lack of space in the anterior chamber.

The above described and other prior attempts to provide an intraocular lens with variable accommodation are generally complex systems. These complex systems are costly and difficult to manufacture and often times impractical to implement in the eye of a human. Therefore, current accommodating lenses provide little accommodating power (about 1 to 2.5 diopters “D”). A true accommodating lens with vastly improved performance should have at least about 4D, preferably at least about 6D or more of accommodating power. Therefore, a need exists for a simple IOL with greater levels of accommodating power that relies only on the forces provided by the human body for operation.

### SUMMARY

The disclosure addresses the shortcomings of prior art lenses and lens assemblies through the use of a novel approach to create large refractive index changes within the refractive system of the AIOL by using air or other gases, with a refractive index of about 1.00, as the medium of low RI. A significant change in refractive power can be achieved with application of minute vertical forces and force changes without the need for movement of the IOL through the optical axis. In addition, the lens is designed to minimize or eliminate posterior capsule opacification (PCO) and capsular bag fibrosis.

In one embodiment, the disclosure relates to an intraocular lens comprising: an optical element adapted to be implanted within a human eye, wherein the optical element has a posterior lens forming a refractive surface with a positive diopter and a gas chamber containing gas adjacent to a polymeric membrane forming a refractive surface of a negative power.

In one embodiment, the disclosure relates to a lens for implantation in the human eye comprising a refractive surface with a positive diopter and a gas chamber containing gas adjacent to a polymeric membrane forming a refractive surface of a negative power.

In one embodiment, the disclosure relates to an accommodating intraocular lens assembly for implantation into a generally circular inner surface of an eye, comprising:
(a) a lens body having an optic and having at least two optic hinge portions, a polymeric membrane and a gas chamber containing gas, wherein the gas chamber is adjacent to the polymeric membrane;
(b) at least two haptics, each haptic having a haptic hinge portion pivotally connected to said optic hinge portions, said haptics spaced apart from each other generally radially away from the optic, adapted to engage the generally circular inner surface of the eye for holding the lens in the eye.

In one embodiment, the intraocular lens (IOL) of the disclosure comprises a system with a posterior lens forming a refractive surface with a positive diopter and an air chamber adjacent to a polymeric membrane forming a refractive surface of a negative power.

Some of the advantages of the lenses and methods disclosed herein include but are not limited to:
1. Stable and predictable refraction since no fluids are used;
2. The AIOL responds to minute forces. This allows a change of curvature with minimal forces from ciliary muscles and hence significantly larger diopter changes;
3. The design is relatively simple and similar to that of conventional IOLs;
4. The AIOL disclosed herein can be injected through a small incision;
5. The capsular bag is kept open after removal of the natural lens. This prevents PCO and fibrosis; and
6. Square edges may be incorporated into the design to prevent posterior capsule opacification (PCO).

In one embodiment, the lens affords true accommodation and large diopter changes.

In one embodiment, the lens and methods disclosed herein are directed to substitute for a natural lens after its removal from the eye, not only by enabling the eye to see after implantation of the assembly, but also by enabling it to accommodate and thereby bring into focus objects located at a continuum of distances. In order to achieve the latter, the assembly is designed to be fixed in the posterior chamber, with the resilient body axially abutting the posterior capsule.

The lens assembly of the disclosure utilizes the natural compression and relaxation of the capsular unit to impart an axial force on the resilient body in order to cause it to act as a lens whose radius of curvature, and therefore the refractive power it provides, varies depending on the magnitude of the force. In this way, the lens assembly cooperates with the natural operation of the eye to accommodate and enable the eye to clearly see objects at different distances.

The haptics element of the lens assembly disclosed herein may adopt any of a variety of designs known in the art, e.g. it may be curved or it may be in the form of a plate. In addition, the haptics element may be completely transparent or opaque.

The haptics element of the lens assembly disclosed herein may be made of a variety of possible rigid materials suitable for invasive medical use and known in the art to be used in the formation of haptics.

The advantages provided by the accommodating lens assembly disclosed herein are many. The lens assembly does not need to conform to the size or shape of the capsule, and is therefore free to take on a larger variety of designs. Furthermore, the capsule is sometimes damaged during the surgery to remove the natural lens, but the lens assembly disclosed herein does not require that the capsule be completely intact in the form of a bag but merely that it remain reliably connected as part of the capsular unit.

Another advantage arising from the lens assembly being positioned outside of the posterior capsule is that it remains unaffected by the permanent and unpredictable constriction that the capsule inevitably undergoes due to scarring following the surgery for removal of the natural lens.

In addition to the above, the lens assembly disclosed herein offers advantages such as a simple and inexpensive construction. The lens assembly disclosed herein also provides the ability to accommodate within a vast range of refractive power, including the full range provided by the natural eye and much more if needed in case of other eye diseases such as age related macular degeneration (AMD). Also, the lens assembly provides means for varying its sensitivity in response to the force applied by the capsular unit.

From the foregoing context it will be appreciated that improvements in the eye care industry can be made with respect to correction of vision such as myopia, hyperopia, presbyopia, replacement of bifocal vision following cataract extraction and treatment of retinal dysfunction such as macular degeneration.

### BRIEF DESCRIPTION OF THE DRAWINGS

FIG. 1 is a schematic of a representative human eye.

FIG. 2 is a front view of a representative embodiment of a spectacle-free accommodating lens or Lens Assembly (SFAL) in accordance with the disclosure.

FIGS. 3A and 3B are representative depictions of a cross-sectional view of a spectacle-free accommodating lens or Lens Assembly (SFAL). FIGS. 3A and 3B depict the changes in diopter induced by accommodation forces. FIG. 3A is a representative depiction of the lens in an unaccommodated state with a diopter value of 20D. FIG. 3B is a representative depiction of the lens in an accommodated state with a diopter value of 26D.

FIGS. 4A-4D are representative depictions of one embodiment of one configuration of a lens/lens assembly with a gas pocket.

FIGS. 5A-5C are representative depictions of one embodiment of one configuration of a lens/lens assembly with a gas pocket.

FIGS. 6A-6D are representative depictions of one embodiment of one configuration of a lens/lens assembly with a gas pocket.

FIGS. 7A-7B are representative depictions of one embodiment of one configuration of a lens/lens assembly with a gas pocket.

FIGS. 8A-8C are representative depictions of one embodiment of one configuration of a lens/lens assembly with a gas pocket.

FIGS. 9A-9D are representative depictions of one embodiment of one configuration of a lens/lens assembly with a gas pocket.

FIGS. 10A-10C are representative depictions of one embodiment of one configuration of a lens/lens assembly with a gas pocket.

FIGS. 11A-11D are representative depictions of one embodiment of one configuration of a lens/lens assembly with a gas pocket.

FIGS. 12A-12C are representative depictions of one embodiment of one configuration of a lens/lens assembly with a gas pocket.

FIGS. 13A-13B are representative depictions of one embodiment of one configuration of a lens/lens assembly with a gas pocket.

FIGS. 14A-14D are representative depictions of one embodiment of one configuration of a lens/lens assembly with a gas pocket.

FIGS. 15A-15C are representative depictions of one embodiment of one configuration of a lens/lens assembly with a gas pocket.

FIGS. 16A-16C are representative depictions of one embodiment of one configuration of a lens/lens assembly with a gas pocket.

FIGS. 17A-17D are representative depictions of one embodiment of one configuration of a lens/lens assembly with a gas pocket.

FIGS. 18A-18E are representative depictions of one embodiment of one configuration of a lens/lens assembly with a gas pocket.

FIGS. 19A-19E are representative depictions of one embodiment of one configuration of a lens/lens assembly with a gas pocket.

FIGS. 20A-20E are representative depictions of one embodiment of one configuration of a lens/lens assembly with a gas pocket.

FIGS. 21A-21C are representative depictions of a model eye set-up for testing an intraocular lens/lens assembly.

FIG. 22 is a graph plotting model eye refraction against anterior lens curvature.

### DETAILED DESCRIPTION

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, diopter value and refractive indexes.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “about” when referring to a measurable value such as an amount, a temporal duration, and the like, is meant to encompass variations of +/−20%, more preferably +/−10%, even more preferably +/−5% from the specified value, as such variations are appropriate to reproduce the disclosed methods and products.

As used herein, an “accommodating IOL” has both an aspheric design and flexible “haptics,” which are the supporting legs that hold the IOL in place inside the eye. These flexible legs allow the accommodating IOL to move forward slightly when you look at near objects, which increases the focusing power of the eye enough to provide better near vision than a conventional monofocal lens.

As used herein, the term “capsular unit” refers to the posterior capsule, the zonules, and the ciliary body that are interconnected and act in unison forming, a cable whose varying tension provides the axial force applied to and utilized by the lens assembly to achieve accommodation.

As used herein, the term diopter (D) refers to the reciprocal of the focal length of a lens in meters. For example, a 10 D lens brings parallel rays of light to a focus at ( 1/10) meter. After a patient's natural crystalline lens has been surgically removed, surgeons usually follow a formula, based on their own personal preference, to calculate a desirable diopter power (D) for the selection of an IOL for the patient to correct the patient's preoperational refractive error. For example, a myopia patient with −10 D undergoes cataract surgery and IOL implantation; the patient can see at a distance well enough even without glasses. This is because the surgeon has taken the patient's −10 D near-sightedness into account when choosing an IOL for the patient.

As used herein, the term “gas chamber” refers to a space or cavity or pocket that contains gas. The gas chamber may be completely filled or partially filled with a gas. In one embodiment, the gas chamber can contain a mixture of two or more gases. In one embodiment, the gas chamber may completely surround a structure, including but not limited to a membrane or may partially surround a membrane.

As used herein, an “intraocular lens” refers to a polymeric aphakic or aphakic (also referred to in the art as pseudophakic), vision-correcting device that may be implanted into a patient's eye. Phakic lenses are used to correct refractive errors such as myopia (near-sightedness), hyperopia (far-sightedness) and astigmatism (blurred vision due to poor light focusing on the retina due to an irregularly shaped cornea or, in some instances, an irregularly shaped natural lens). The natural lens remains in place when a phakic lens is implanted while the lens is removed prior to implantation of pseudophakic lens. An aphakic or pseudophakic lens is inserted in the eye subsequent to removal of the natural lens due to disease, most often a cataract; that is, clouding of the natural lens. Either type of lens may be implanted in the anterior chamber in front of the iris or in the posterior chamber behind the iris and in front of the natural lens or in the region where the natural lens was before removal. While intraocular lenses may be “hard,” that is relatively inflexible, or “soft,” i.e., relatively flexible but not foldable, for the purpose of this invention the presently preferred lens is a foldable acrylic polymer lens A foldable lens is one that is sufficiently flexible that it can be folded into a smaller configuration to permit its implantation into the eye through a much smaller incision that is necessary for hard or soft lenses. That is, while hard and soft lenses may require a 6 mm or larger incision, a foldable lens usually requires only a 3 mm or even smaller incision. U.S. Pat. No. 7,789,509 to Mentak, U.S. Pat. No. 6,281,319 to Mentak, U.S. Pat. No. 6,635,731 to Mentak, U.S. Pat. No. 6,635,732 to Mentak, and U.S. Pat. No. 7,083,645 to Mentak, U.S. Pat. No. 7,789,509 to Mentak el al., and U.S. Pat. No. 7,399,811 also to Mentak et al. are all incorporated herein by reference in their entirety.

As used herein, the term “shape changing optical element” refers to an optical element that is made of material that enables the optical element to alter its shape, e.g. become one of more spherical in shape, thicker or focus on a closer object, or become more ovoid in shape, thinner or focus on a more distant object and thus alter the optical element's respective optics (alter the diopters of the resulting optical element)

As used herein, the term “accommodating shape” refers to the shape of the optical element when at least one of the tensioning of the ciliary muscle of the mammalian eye, the zonules of the mammalian eye and a change in the vitreous pressure in the eye effect equatorial or polar distention of the capsular bag to effect a focusing upon a closer object. An accommodating shape is generally more spherical than the disaccommodating shape.

As used herein, the term “disaccommodating shape” refers to the shape of the optical element when at least one of the relaxation of the ciliary muscle of the mammalian eye, the zonules of the mammalian eye and a change in the vitreous pressure in the eye and a concomitant change to a more ovoid shaping of the capsular bag to effect a focusing upon a more distant object. A disaccommodating shape is generally more ovoid than the accommodating shape.

As used herein, the refractive index or index of refraction of a material is a dimensionless number that describes how light propagates through that medium. It is defined as: where c is the speed of light in vacuum and ν is the phase velocity of light in the medium. For example, the refractive index of water is 1.333, meaning that light travels 1.333 times faster in vacuum than in the water.

As used herein, “optical component,” “optical assembly” or “optical subassembly” shall mean a portion of, or a completed, ophthalmic device, assembly or subassembly. Non-limiting examples of optical components include lens bodies, optic bodies, haptics, IOL components.

As used herein, “optical polymer” refers to a polymer that is suitable for implantation into a patient's eye and that is capable of addressing ophthalmic conditions of the lens of the eye such as, without limitation, myopia, hyperopia, astigmatism and cataracts. In general such a polymer will be biocompatible, i.e., it will not cause any inflammatory, immunogenic, or toxic condition when implanted, it will form a clear, transparent, colorless (unless intentionally colored for a particular application) film-like membrane, and it will have a refractive index greater than about 1.4, preferably greater than about 1.5 and presently most preferably greater than about 1.55.

The apparatuses and methods disclosed herein will now be described more fully hereinafter with reference to the accompanying drawings, in which embodiments of the disclosure are shown. The apparatuses and methods disclosed herein may, however, be embodied in many different forms and should not be construed as limited to the embodiments set forth herein. Rather, these embodiments are provided so that the disclosure will be thorough and complete and will fully convey the scope of the invention to those skilled in the art.

It will be appreciated by those skilled in the art that the set of features and/or capabilities may be readily adapted within the context of a standalone weapons sight, front-mount or rear-mount clip-on weapons site, and other permutations of filed deployed optical weapons sights. Further, it will be appreciated by those skilled in the art that various combinations of features and capabilities may be incorporated into add-on modules for retrofitting existing fixed or variable weapons sights of any variety.

It will be understood that when an element or layer is referred to as being “on,” “connected to” or “coupled to” another element or layer, it can be directly on, connected or coupled to the other element or layer. Alternatively, intervening elements or layers may be present. In contrast, when an element is referred to as being “directly on,” “directly connected to” or “directly coupled to” another element or layer, there are no intervening elements or layers present.

Like numbers refer to like elements throughout. As used herein, the term “and/or” includes any and all combinations of one or more of the associated listed items.

It will be understood that, although the terms first, second, etc., may be used herein to describe various elements, components, regions, and/or sections, these elements, components, regions, and/or sections should not be limited by these terms. These terms are only used to distinguish one element, component, region, or section from another element, component, region, or section. Thus, a first element, component, region, or section discussed below could be termed a second element, component, region, or section without departing from the disclosure.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90° or at other orientations) and the spatially relative descriptors used herein interpreted accordingly.

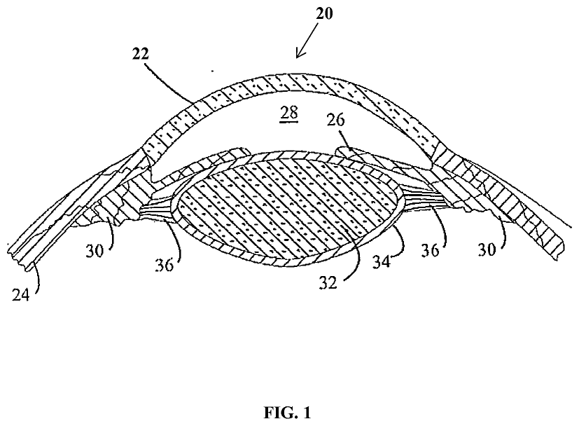

Turning now to FIG. 1 there will be seen a partial cross-sectional view of an anterior segment of a human eye 20. Vision in humans is provided by a first convex/concave lens known as a cornea 22. This segment is partially spherical and is transparent to light. The cornea 22 is connected at its perimeter to a generally spherical exterior body of the eye known as a sclera 24. An iris 26 is positioned within an anterior chamber of the eye 28 and serves to vary the amount of light permitted to pass into the eye structure. The iris 26 extends into and is joined with a muscular structure known as the ciliary body or muscle 30 which extends peripherally about an interior portion of the eye. A natural crystalline lens 32 is positioned behind the iris 26 and is enrobed by a capsular membrane or bag 34. The natural crystalline lens 32 approximates an ellipse in cross-section and is circular when viewed along a line of sight. Zonula of Zinn 36 extend between the ciliary muscle 30 and an equator position of the capsular bag 34. A hyloid face, not shown, extends across the posterior surface of the lens 32 and isolates the forward segment of the eye from a vitreous chamber filled with clear vitreous humor.

Light is focused by the human eye by being refracted through the cornea and then refracted again through the bi-convex natural crystalline lens and is focused on a retina at the base of the eye. Bifocal vision from infinity to 250 millimeters is accommodated by varying the shape of the natural crystalline lens 32. More specifically, images at infinity are focused by the ciliary muscle 30 relaxing which permits their peripheral expansion and thus tensioning the zonula 36. Tension of the zonula draws the equator of the capsular bag radially outward and foreshortens the thickness of the lens body 32, providing for distance vision. In contrast, near vision is accommodated in a human eye by the ciliary muscles contracting which releases tension on the zonula allowing the lens body 32 to thicken into its natural state and thus focusing near objects upon the retina for transmission to the brain by the optic nerve.

A human eye adapts readily to variations in focal length and seamlessly enables a human to view objects at infinity as well as near vision instantly without conscious accommodation. Notwithstanding the perfect vision enjoyed by a majority of the population, an inability to view objects at infinity, or myopia, is frequently′ encountered. This visual impairment can be corrected by refractive lens held by glasses, wearing contact lens or refractive surgery. In addition, certain humans do not focus near vision well. This is known as hyperopia and their vision can also be corrected by conventional refractive techniques. In certain instance of severe lack of accommodation these conventional procedures become undesirable and alternative procedures are needed.

Although a youth of ten years in age has an ability to change the dioptic power by fourteen diopters, this ability gradually decreases with age and by fifty or so the ability of the human eye to accommodate variation in focal length becomes essentially zero. This condition is referred to by presbyopia and a patient often requires correction for both near vision and far vision. This can be achieved by wearing bifocal glasses or contacts or undergoing refractive surgery for distance and wearing glasses for reading purposes.

In addition to the foregoing more conventional limitations on 20/20 vision, in instances of juvenile disease, trauma, and more frequently through age, the natural crystalline lens 32 becomes rigid and opaque to the passage of light. This condition is referred to as a cataract which can be corrected by removal of the lens 32 by a number of techniques, however, the most commonly perform surgery is known as extracapsular extraction. In this procedure, an annular opening is fashioned about the anterior visual center of the lens, centered by the iris, and then emulsifying and aspirating the hardened lens material. At least one procedure for phacoemulsification, irrigation and aspiration is disclosed in a U.S. Pat. No. 5,154,696.

Once the natural crystalline lens is removed a bi-convex, fixed focal length optic, of about six millimeters in diameter, is typically fitted into the capsular bag and held in position by radially extending haptics. Although cataract surgery and insertion of an intraocular lens is the most frequently performed surgical procedure in the United States, and has achieved a considerable degree of sophistication and success, an intraocular lens selected with a diopter to achieve far vision and near vision must be corrected by wearing reading glasses.

Finally, retinal disease or damage can impair human vision and one form is known as macular degeneration which usually occurs with advance in age. The symptom of macular degeneration can be alleviated, to a degree, by providing high diopters in the 30 to 70 range such that the rods and cones available to receive sight are utilize to their fullest.

Continuous tear circular capsulotomy, or capsulorhexis, involves tearing the anterior capsule along a generally circular tear line in such a way as to form a relatively smooth-edged circular opening in the center of the anterior capsule. The cataract is removed from the natural lens capsule through this opening. After completion of this surgical procedure, the eye includes an optically clear anterior cornea 22, an opaque sclera 24 on the inner side of which is the retina of the eye, an iris 26, a capsular bag 34 behind the iris, and a vitreous cavity behind the capsular bag filled with the gel-like vitreous humor. The capsular bag 34 is the structure of the natural lens of the eye which remains intact within the eye after the continuous tear circular tear capsulorhexis has been performed and the natural lens matrix has been removed from the natural lens.

The capsular bag 34 includes ark annular anterior capsular remnant or rim and an elastic posterior capsule which are joined along the perimeter of the bag to form an annular crevice-like cul-de-sac between rim and posterior capsule. The capsular rim is the remnant of the anterior capsule of the natural lens which remains after capsulorhexis has been performed on the natural lens. This rim circumferentially surrounds a central, generally round anterior opening (capsulotomy) in the capsular bag through which the natural lens matrix was previously removed from the natural lens. The capsular bag 34 is secured about its perimeter to the ciliary muscle 30 of the eye by zonules 30.

Natural accommodation in a normal human eye having a normal human crystalline lens involves automatic contraction or constriction and relaxation of the ciliary muscle of the eye by the brain in response to looking at objects at different distances. Ciliary muscle relaxation, which is the normal state of the muscle, shapes the human crystalline lens for distant vision. Ciliary muscle contraction shapes the human crystalline lens for near vision. The brain-induced change from distant vision to near vision is referred to as accommodation.

In order to understand the lenses and methods disclosed herein and to see how it may be carried out in practice, a preferred embodiment will now be described, by way of non-limiting example only, with reference to the accompanying drawings.

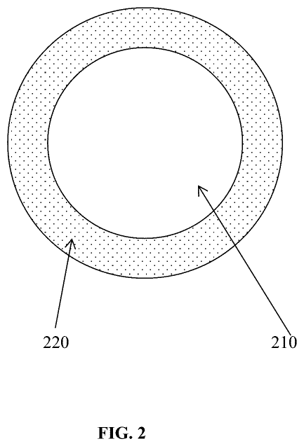

FIG. 2 displays a front view of a spectacle-free accommodating lens (SFAL) as one embodiment of the lenses disclosed herein. FIG. 2 displays a lens assembly comprising an optic chamber 210 and haptic structure 220. It will be understood that the haptic structure in FIG. 2 represents only one possible haptic configuration, there being many others that will readily occur to one skilled in this art in view of this disclosure.

In one embodiment, a scleral lens design has three primary zones: (1) optical, (2) transition and (3) landing. The optical and transition zones provide sagittal depth to the lens for proper vault height, while the third zone—the landing zone-rests gently on the bulbar conjunctiva. This area is also commonly known as the haptics or peripheral curves. Proper alignment of this zone with the sclera is a critical component of a successful scleral lens fit, as any degree of misalignment can negatively impact both comfort and vision.

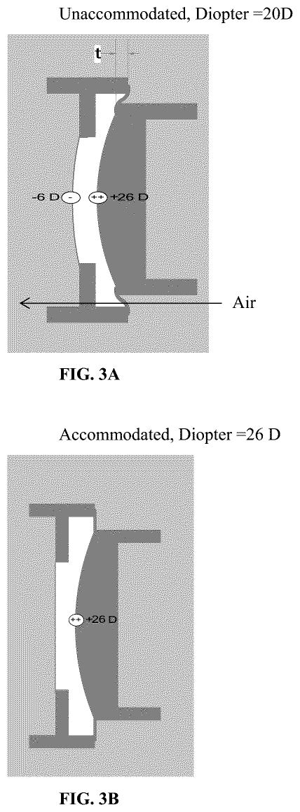

FIGS. 3A and 3B are cross-sectional views of an accommodating lens with a gas chamber showing the relationship between of lens diopter with small changes in the curvature of the anterior membrane. FIG. 3A depicts an accommodating lens with an gas chamber, in this case an air chamber, adjacent to a polymeric membrane in the unaccommodated state (far vision) with a diopter of 20D. FIG. 3B depicts the accommodating lens with an air chamber adjacent to a polymeric membrane in the accommodated state (near vision) with a diopter of 12D. The change in the shape of the anterior surface shown in FIGS. 3A and 3B illustrates the increase refractive power upon changes in haptic pressure.

The interface between the ocular fluid (RI-1.33) and the gas in the chamber (RI=1.00) creates a powerful and sensitive optical system. A very small force is required to cause a significant change in curvature of the membrane separating the two media, which in turn changes the diopter of the lens to provide focus on objects at various distances. The force is transmitted from the ciliary muscles to the system through the haptics. The haptics may be formed into several configuration including C-loop, modified C-loop, square, disk-like, plate, etc.

Changes in haptic pressure are obtained by changes in the deformation of the haptics which, in turn, result from increased pressure of the ciliary muscle or capsular bag. The radius of curvature of the interior envelope changes in response to haptic deformation and capsular forces. That change in radius of curvature in conjunction with the large difference between the RI of air and ocular fluid create substantial dioptric changes for very small ciliary muscle movement(s).

In one embodiment, the accommodating lens includes a central optic. The optic comprises an anterior surface and a posterior surface. The anterior surface and the posterior surface are usually convex, although the shape of these surfaces and size of the optic can be varied depending upon the user's eyesight.

In one embodiment, the lens may further include a resilient body. Resilient body comprises an outer wall that extends radially from the optic. Resilient body is preferably integral and essentially flush with the optic at optic perimeter where the wall joins the optic.

The overall shape of lens in its original resting, non-deformed shape generally conforms to the shape of capsule when capsule is focused to view an object near the viewer. The outer wall of the resilient body cooperates with the optic to form a lens having an overall discoid or saucer-like shape. The lens is of sufficient size so that the optic mildly urges against the anterior wall of the capsule, while the posterior side of lens urges against the posterior wall of the capsule.

The described lens embodiments disclosed herein conform to one of the following basic lens configurations: (a) a lens configuration, hereafter referred to as a posteriorly biased lens configuration, in which the hinges of hinged extended portions and the inner ends of resiliently bendable extended portions are located posteriorly of or approximately in a plane (tip plane) normal to the optic axis and containing the outer tips of the extended portions when the lens occupies its posterior distant vision position against the posterior capsule of the eye, and (b) a lens configuration, hereafter referred to as an anteriorly biased lens configuration, in which the hinges of hinged extended portions and the inner ends of resiliently bendable extended portions are located forwardly of the tip plane when the lens occupies its posterior distant vision position against the posterior capsule of the eye.

Radial compression of a posteriorly biased lens by constriction of the ciliary muscle during accommodation initially urges the lens optic posteriorly against the more dominant anterior forces of the stretched posterior capsule and the increasing vitreous pressure which combine to move the optic forwardly in accommodation against the rearward bias of the compressing lens until the hinges of hinged extended portions or the inner ends of resiliently bendable extended portions move forwardly of the tip plane. Continued radial compression of the lens by ciliary muscle constriction then aids anterior accommodation movement of the lens. Radial compression of an anteriorly biased lens by constriction of the ciliary muscle urges the lens optic anteriorly and thus aids the dominant anterior forces of the stretched posterior capsule and the increasing vitreous pressure throughout the range of lens accommodation.

In another embodiment, the extended portions of a lens embodiment are generally T-shaped haptics each including a haptic plate and a pair of relatively slender resiliently flexible fixation fingers at the outer end of the haptic plate (see FIGS. 3A and 3B). In their normal unstressed state, the two fixation fingers at the outer end of each haptic plate extend laterally outward from opposite edges of the respective haptic plate in the plane of the plate and substantially flush with the radially outer end edge of the plate to form the horizontal “crossbar” of the haptic T-shape. The radially outer end edges of the haptic plates are circularly curved about the central axis of the lens optic to substantially equal radii closely approximating the radius of the interior perimeter of the capsular bag when the ciliary muscle of the eye is relaxed.

During implantation of the lens in the bag, the inner perimetrical wall of the bag deflects the haptic fingers generally radially inward from their normal unstressed positions to arcuate bent configurations in which the radially outer edges of the fingers and the curved outer end edges of the respective haptic plates conform approximately to a common circular curvature closely approximating the curvature of the inner perimetrical wall of the bag. The outer T-ends of the haptics then press lightly against the perimetrical bag wall and are fixated within the bag perimeter during fibrosis to accurately center the implanted lens in the bag with the lens optic aligned with the anterior capsule opening in the bag.

### A. Refractive Index of Gas

In one embodiment, the gas in the gas chamber can have a refractive index of about 1.00. In one embodiment, the gas is selected from the group consisting of: air, helium, hydrogen, and carbon dioxide. In one embodiment, the gas is air.

In one embodiment, the gas in the chamber can be a mixture of two or more gases, with each gas having a refractive index of about 1. Table I provides a list of gases with a refractive index of about 1.

TABLE I
Gases and their respective refractive index

| | Refractive Index |
|---|---|
| Gas | - n - |
| Acetone | 1.001090 |
| Air | 1.000293 |
| Alcohol, ethyl | 1.000878 |
| Alcohol, methyl | 1.000586 |
| Ammonia | 1.000376 |
| Argon | 1.000281 |
| Benzene | 1.001762 |
| Bromine | 1.001132 |
| Carbon dioxide | 1.000449 |
| Carbon disulphide | 1.001481 |
| Carbon monoxide | 1.000338 |
| Chlorine | 1.000773 |
| Chloroform | 1.001450 |
| Ether, ethyl | 1.001533 |
| Ether, methyl | 1.000891 |
| Helium | 1.000035 |
| Hydrochloric acid | 1.000447 |
| Hydrogen | 1.000132 |
| Methane | 1.000444 |
| Nitric oxide | 1.000297 |
| Nitrogen | 1.000298 |
| Nitrous oxide | 1.000516 |
| Oxygen | 1.000271 |
| Pentane | 1.000686 |
| Sulphur dioxide | 1.000686 |
| Water vapor | 1.000261 |

In one embodiment, the gas has a refractive index that is about 33% less than the refractive index of the ocular fluid. In one embodiment, the gas has a refractive index that is from about 20% to about 33% less than the refractive index of the ocular fluid.

In one embodiment, the gas is mixture of two gases with one gas being air and the second gas being a gas other than air with a refractive index of about 1. The mixture can comprise about 50% air or 600/air or 70% air or 80% air or 90% air or 95% air. In one embodiment, the mixture comprises at least 75% air.

In one embodiment, the gas is a mixture of three or more gases, with one gas being air and the second and third gases being gases other than air and having a refractive index of about 1.00.

### B. Gas Chamber

In one embodiment, the gas chamber can be of any suitable shape to provide the desired diopter changes. In one embodiment, the gas chamber is a single chamber. In another embodiment, the lens/lens assembly comprises multiple gas chambers.

In one embodiment, the gas chamber can have a configuration as depicted in any one of FIGS. 3-20. In one embodiment, the gas chamber can reside between multiple layers of a membrane. In one embodiment, the gas chamber can completely surround a membrane or partially surround a membrane.

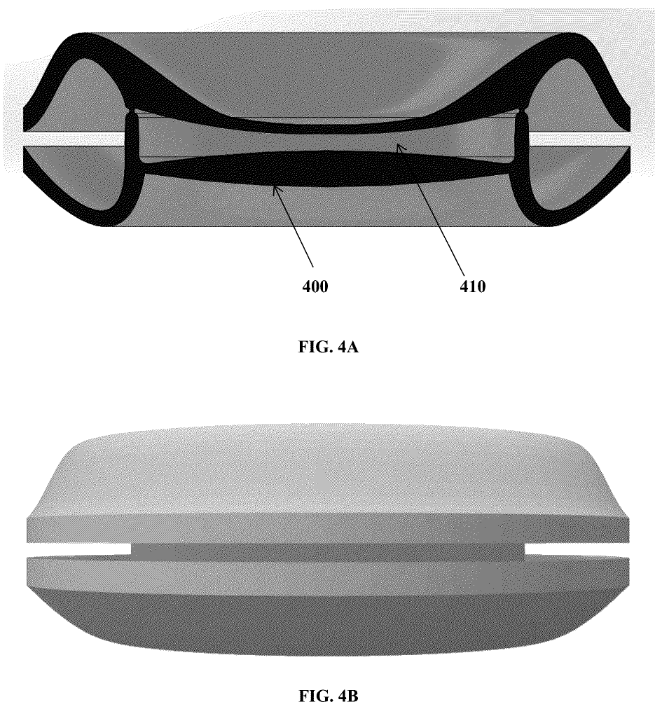

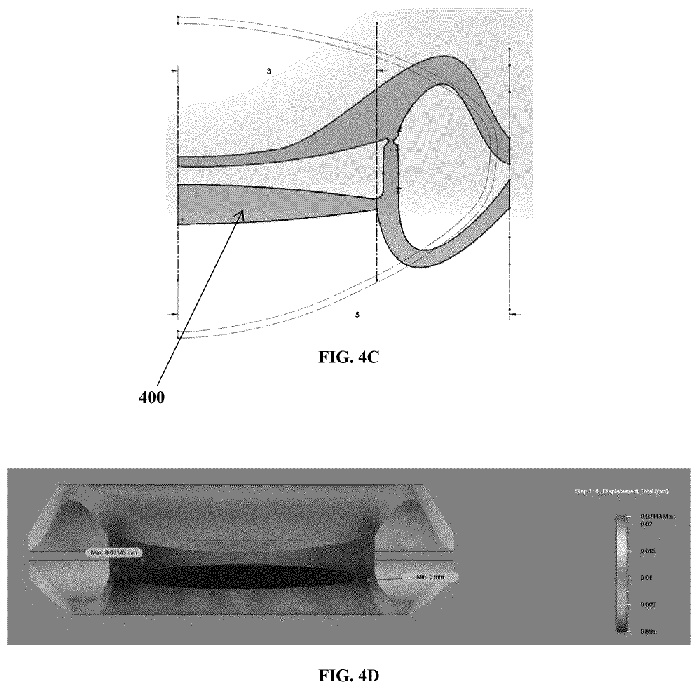

In one embodiment, the gas chamber extends from the top right portion of the lens assembly down toward the center of the lens assembly, and then up toward the top left portion of the lens assembly, and back down toward the left bottom portion of the lens assembly, back toward the center of the lens assembly, and back down toward the right bottom portion of the lens assembly. FIG. 4A provides a representative depiction of the gas chamber described above, where a membrane rests on the bottom or floor of the lens assembly.

In one embodiment, the lens assembly comprises multiple gas chambers. In one embodiment, the lens assembly has a central gas chamber and one or more peripheral gas chambers. In one embodiment, the lens assembly has a central gas chamber, a right peripheral gas chamber and a left peripheral gas chamber.

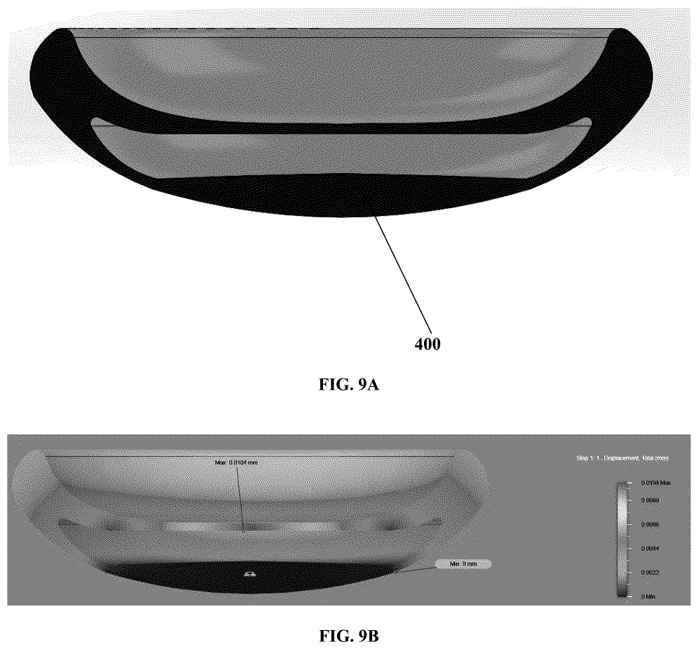

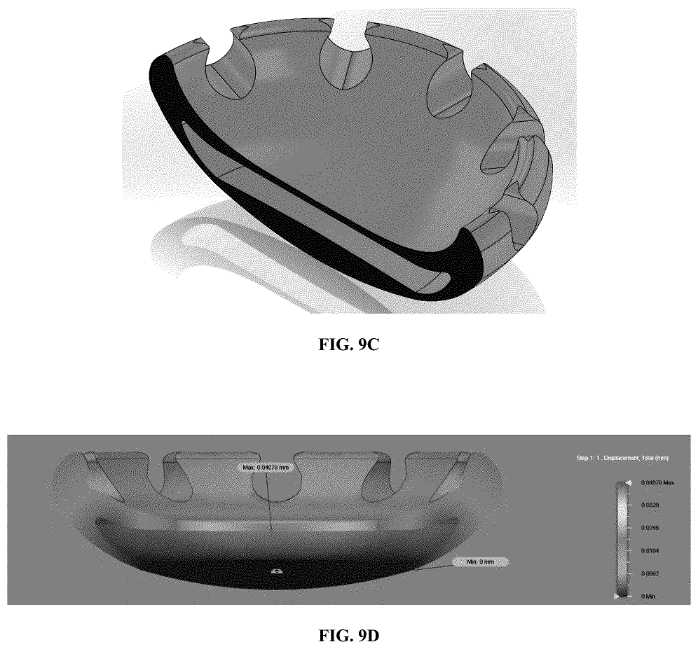

In one embodiment, the gas chamber extends to the bottom of the lens assembly. The gas chamber extends to the floor or bottom of the lens assembly. In another embodiment, the gas chamber extends to the top right and top left portions of the lens assembly. FIG. 9A is a representative depiction of the gas chamber described above.

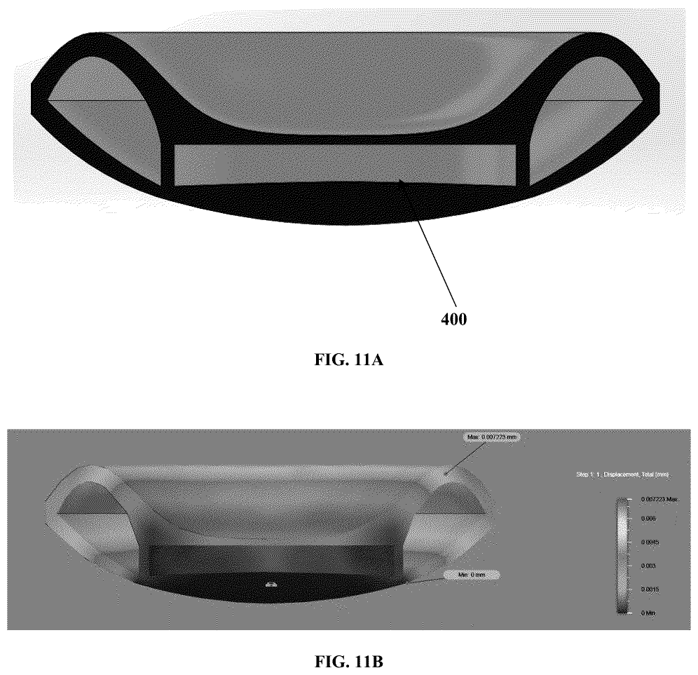

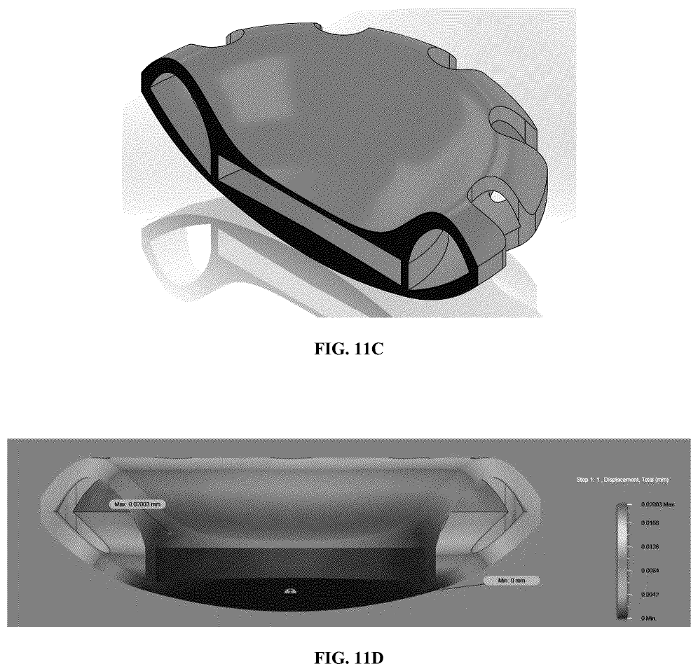

In another embodiment, the gas chamber extends to the bottom of the lens assembly and can surround membrane that is located at the top right and top left portions of the lens assembly. FIG. 11A is a representative depiction of the gas chamber described above.

### C. Diopter Change

In one embodiment, the lenses disclosed herein have accommodating power of 4D or 5D or 6D or 7D or 8D or 9D or 10D or 11D or 12D or even greater than 12D.

In one embodiment, diopter changes of the lenses and methods disclosed herein are from 4 to 12 diopters or from 4 to 10 diopters or from 4 to 8 diopters or from 4 to 6 diopters. In another embodiment, diopter changes of the lenses and methods disclosed herein are from 6 to 12 diopters or from 8 to 12 diopters or from 10 to 12 diopters.

### D. Materials

The materials chosen to practice the methods disclosed herein will be readily apparent to one skilled in the art. In one embodiment, haptic materials may include PMMA, PVDF, PP, or other polymer. In another embodiment, optic chamber materials can include hydrophobic acrylic polymers or copolymers (HAC), hydrophilic acrylic polymers or copolymers, silicone polymers or copolymers (PDMS) or other polymers. Preferred polymers include PDMS or HAC.

Monomers suitable for the preparation of hydrophobic acrylic polymers cover a wide range of structures including, but not limited to: phenoxyethylacrylate, 2-phenylethylacrylate, styrene, methylacrylate, ethylacrylate, hexylmethacrylate, laurylmethacrylate, stearylacrylate, methylmethacrylate, phenoxyethylmethacrylate, 2-phenylethylmethacrylate, laurylmethacrylate, stearylmethacrylate, alkylacrylate derivatives and alkylmethacrylate derivatives.

In one embodiment, the polymeric membrane is composed of silicone.

### Additional Embodiments

Reference is now made to FIGS. 4A-4D, which illustrate an accommodating intraocular lens with a gas chamber, in this example an air chamber 400, constructed and operative in accordance with an embodiment of the disclosure. In this embodiment, the lens is in an accommodated state where capsular equator stretch or tension causes haptics to move together, thereby stretching/flattening the membrane 410 and resulting in dis-accommodation.

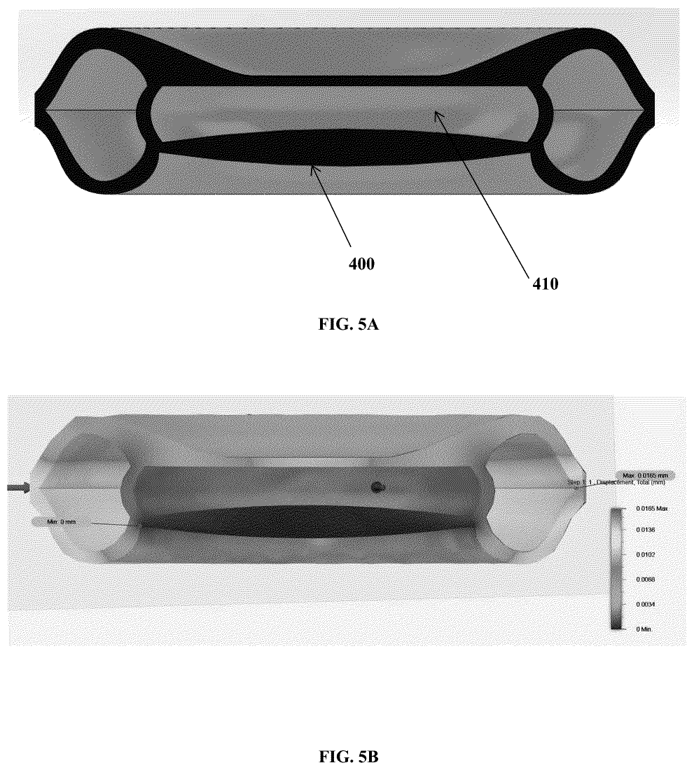

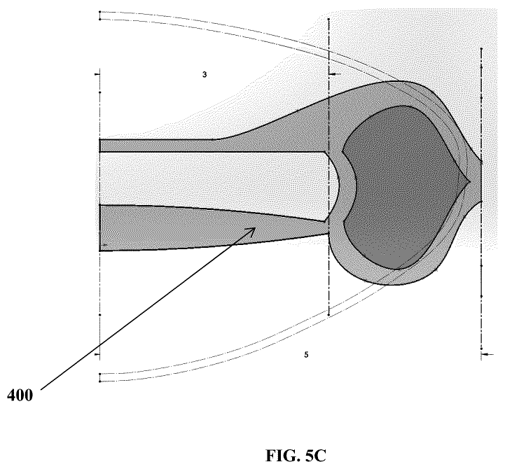

Reference is now made to FIGS. 5A-5C, which illustrate an accommodating intraocular lens with a gas chamber, in this example an air chamber 400, constructed and operative in accordance with an embodiment of the disclosure. In this embodiment, dis-accommodated molded state is shown where the capsular diameter reduction during accommodation causes concave movement of membrane 410, thereby resulting in accommodation. In one embodiment, negative pressure can be applied to the air pocket making the lens more sensitive to capsular forces.

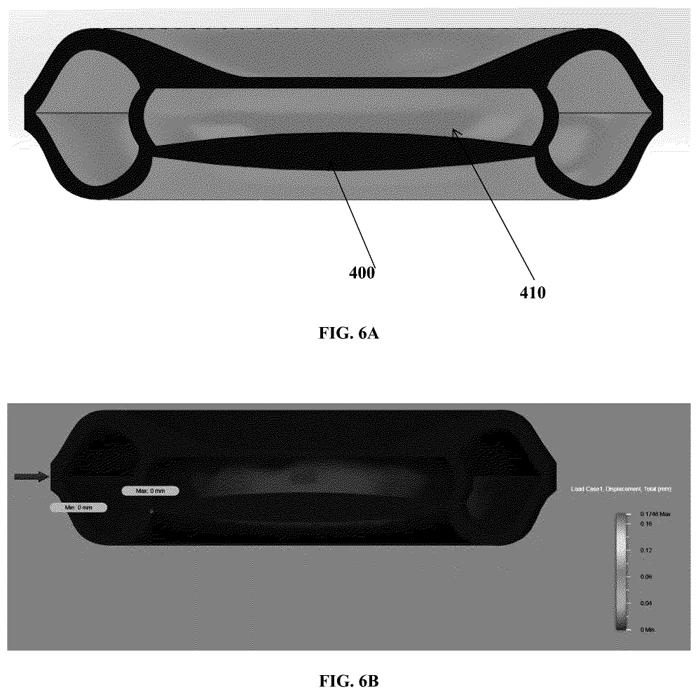

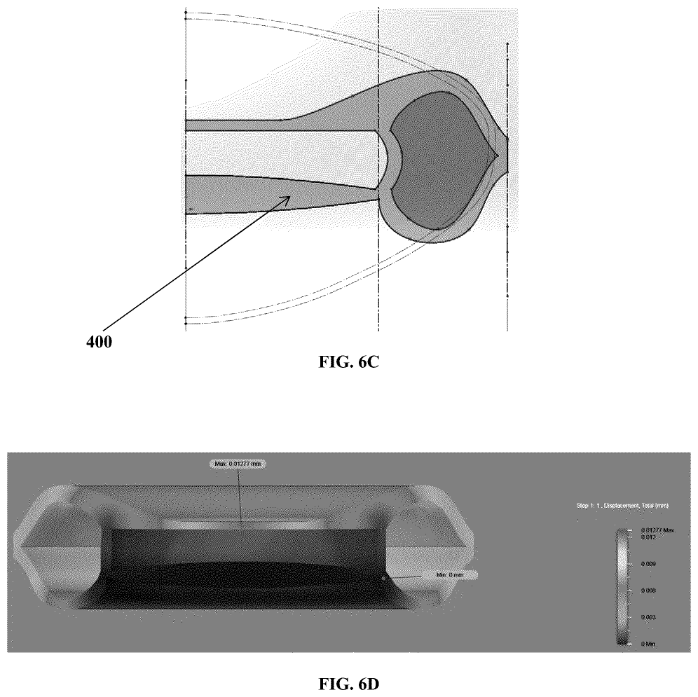

Reference is now made to FIGS. 6A-6D, which illustrate an accommodating intraocular lens with a gas chamber, in this example an air chamber 400, constructed and operative in accordance with an embodiment of the disclosure. In this embodiment, dis-accommodated/molded state is shown where capsular bag forces during accommodation cause concave movement of membrane, resulting in accommodation.

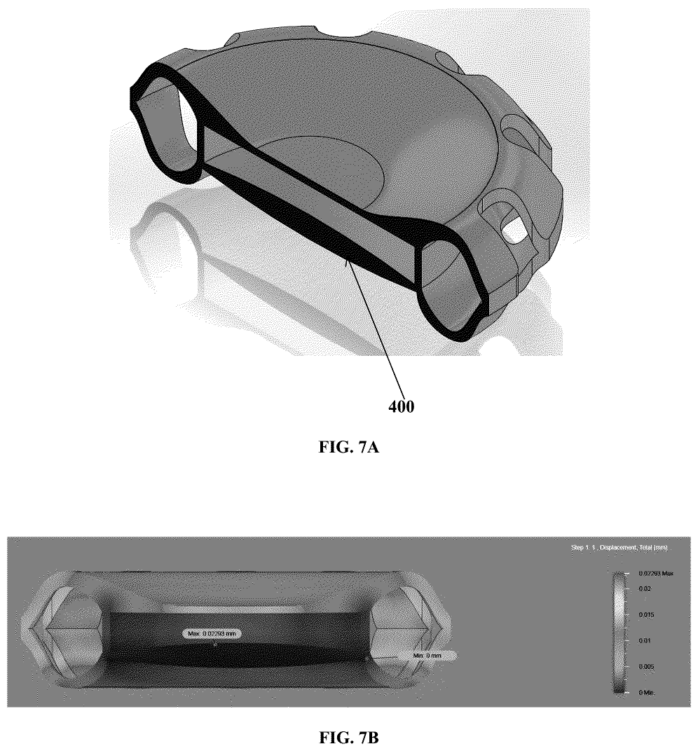

Reference is now made to FIGS. 7A-7B, which illustrate an accommodating intraocular lens with a gas chamber, in this example an air chamber 400, constructed and operative in accordance with an embodiment of the disclosure. In this embodiment, the haptics promote greater membrane concavity by reducing the forces required to compress the lens during accommodation.

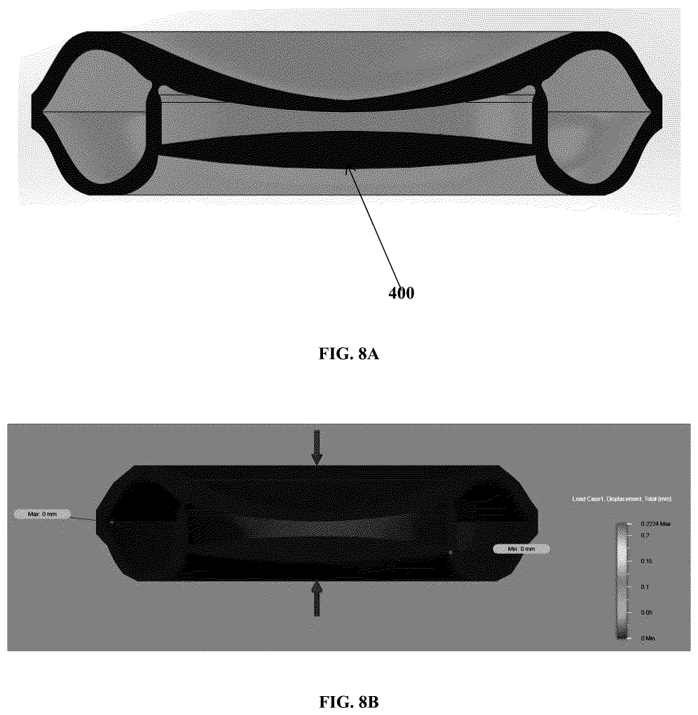

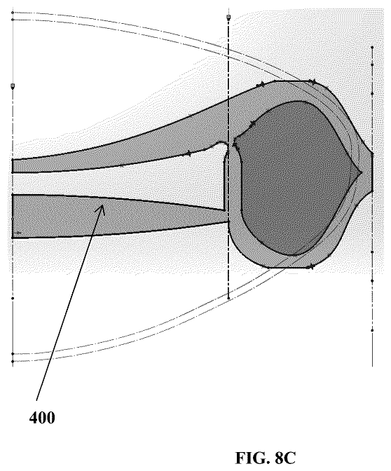

Reference is now made to FIGS. 8A-8C, which illustrate an accommodating intraocular lens with a gas chamber, in this example an air chamber 400, constructed and operative in accordance with an embodiment of the disclosure. In this embodiment, accommodated/molded state is shown where the capsular stretch/tension causes haptics to move together, stretching the membrane and resulting in dis-accommodation.

Reference is now made to FIGS. 9A-9D, which illustrate an accommodating intraocular lens with a gas chamber, in this example an air chamber 400, constructed and operative in accordance with an embodiment of the disclosure. In this embodiment, the optic is seated against posterior bag for stability (9A). There is more direct force transmission from capsular equator and good membrane movement. As shown in FIGS. 9C-9D, peripheral haptics promote greater membrane concavity by reducing the forces required to compress the lens during accommodation.

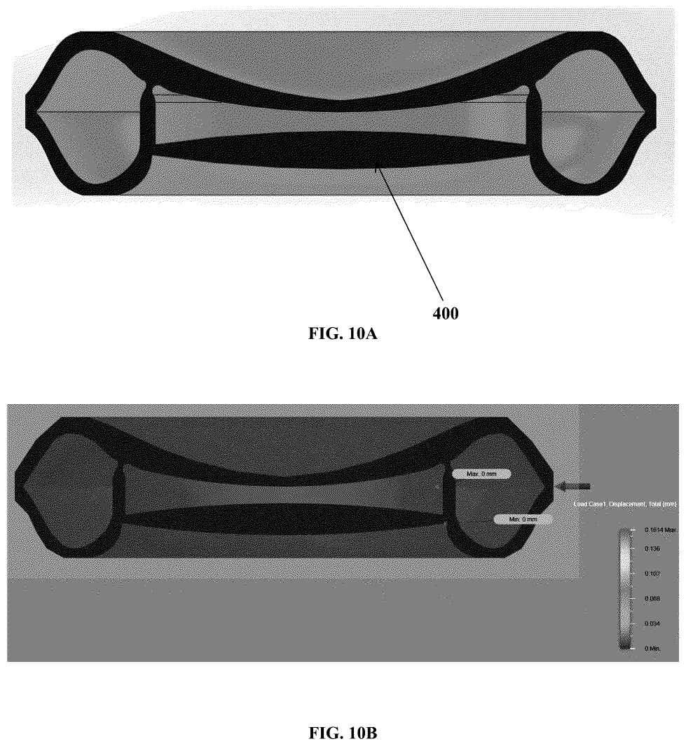

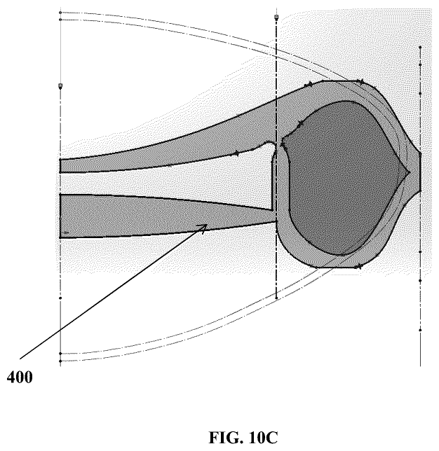

Reference is now made to FIGS. 10A-10C, which illustrate an accommodating intraocular lens with a gas chamber, in this example an air chamber 400, constructed and operative in accordance with an embodiment of the disclosure. In this embodiment, dis-accommodated/molded state is shown where capsular diameter reduction during accommodation causes further concave movement of membrane, resulting in accommodation. In one embodiment, negative pressurization of air pocket could provide a design that is more sensitive to forces.

Reference is now made to FIGS. 11A-11D, which illustrate an accommodating intraocular lens with a gas chamber, in this example an air chamber 400, constructed and operative in accordance with an embodiment of the disclosure. In this embodiment, optic is seated against posterior bag for stability. As shown in FIGS. 11C-11D, peripheral haptics promote greater membrane concavity by reducing the forces required to compress the lens during accommodation.

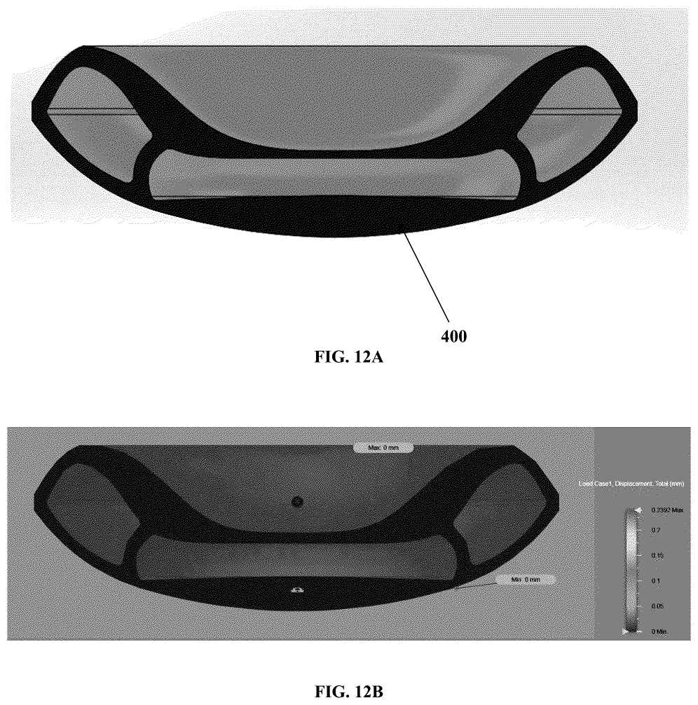

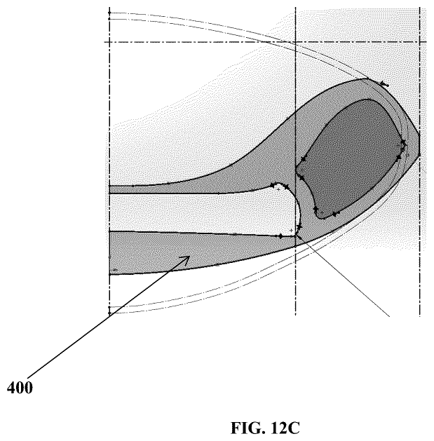

Reference is now made to FIGS. 12A-12C, which illustrate an accommodating intraocular lens with a gas chamber, in this example an air chamber 400, constructed and operative in accordance with an embodiment of the disclosure. In this embodiment, dis-accommodated/molded state is shown where capsular bag forces during accommodation cause concave movement of membrane resulting in accommodation.

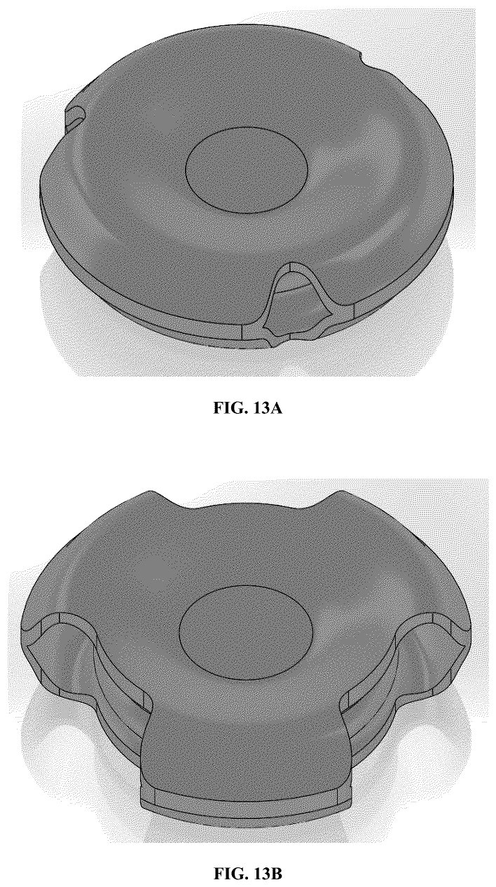

Reference is now made to FIGS. 13A-13B, which illustrate an accommodating intraocular lens constructed and operative in accordance with an embodiment of the disclosure. In this embodiment, cutouts can be added to the variations of the lens/lens assemblies discussed above. The cut-outs on the peripheral edges can aid in regulating forces and pressure.

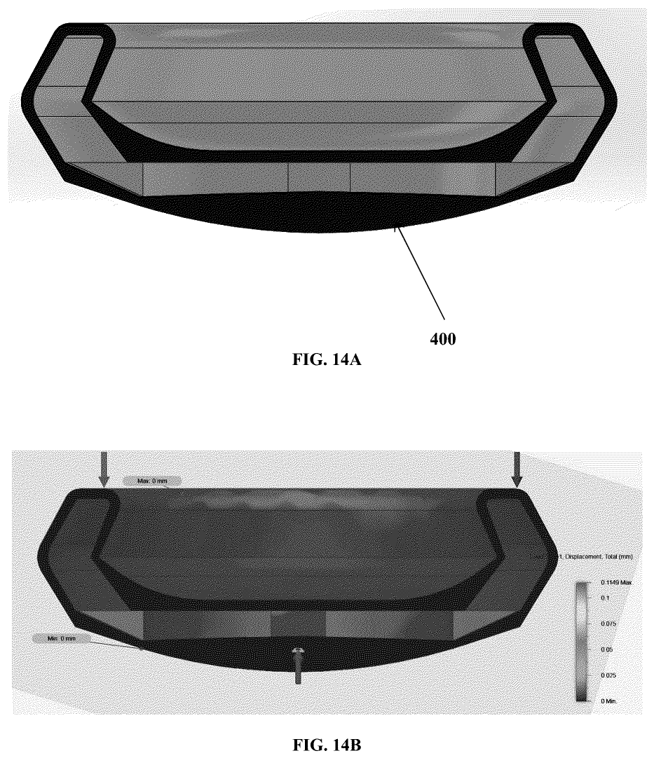

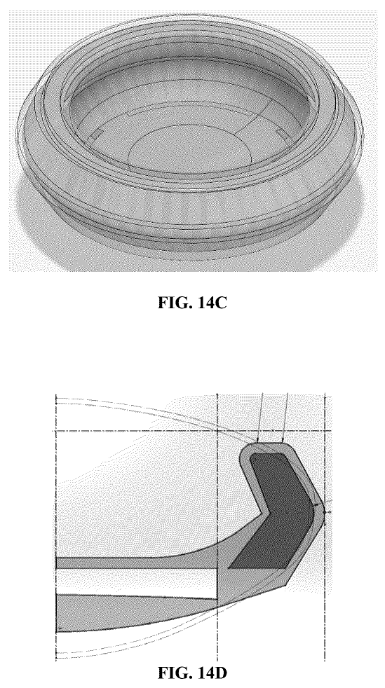

Reference is now made to FIGS. 14A-14C, which illustrate an accommodating intraocular lens with a gas chamber, in this example an air chamber 400, constructed and operative in accordance with an embodiment of the disclosure. In this embodiment, capsular equator stretch/tension causes hinging peripheral air pocket to compress, moving air to a central air pocket, thereby bulging the central membrane and resulting in dis-accommodation. In this embodiment, a smooth transition from peripheral haptic to central membrane was added, which should transmit mechanical force during hinging to aid in membrane bulge.

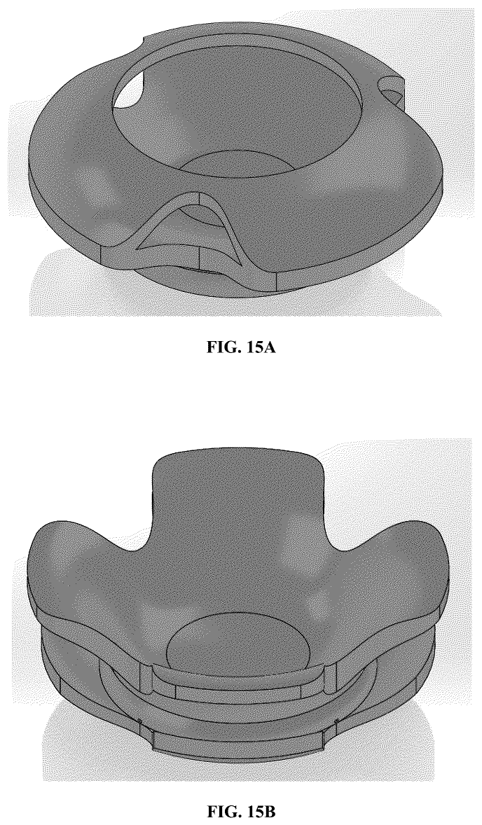

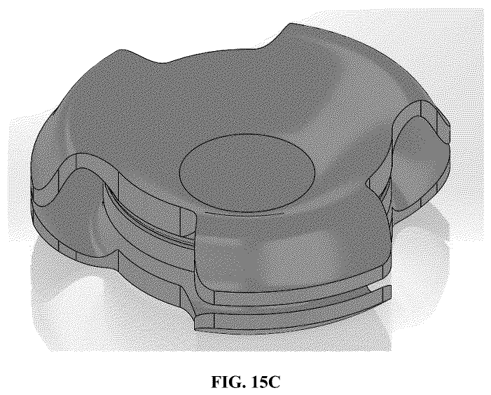

Reference is now made to FIGS. 15A-15C, which illustrate an accommodating intraocular lens with a gas chamber, in this example an air chamber 400, constructed and operative in accordance with an embodiment of the disclosure. In this embodiment, various configurations are provided that have been skeletonized. The configurations are designed for ease of implantation or modulation of pressure or modulation of force.

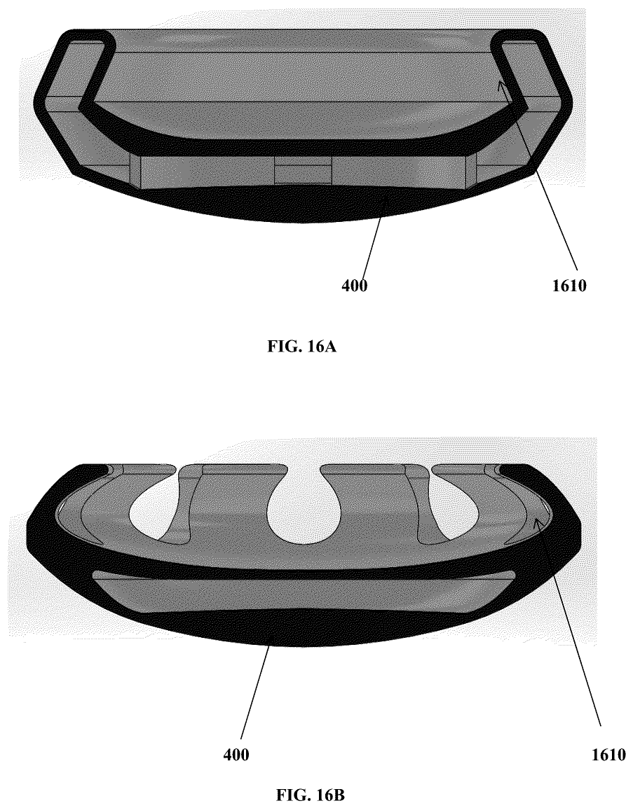

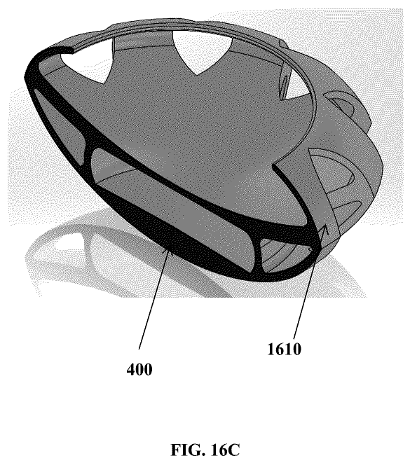

Reference is now made to FIGS. 16A-16C, which illustrate an accommodating intraocular lens with a gas chamber, in this example an air chamber 400, constructed and operative in accordance with an embodiment of the disclosure. In one embodiment, various configurations are provided with an overhang 1610. The overhang 1610 helps to maintain the capsular bag in an open/receptive state.

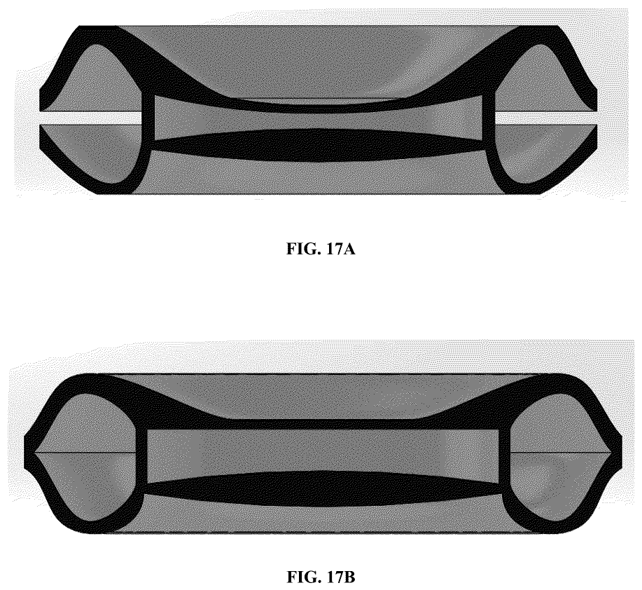

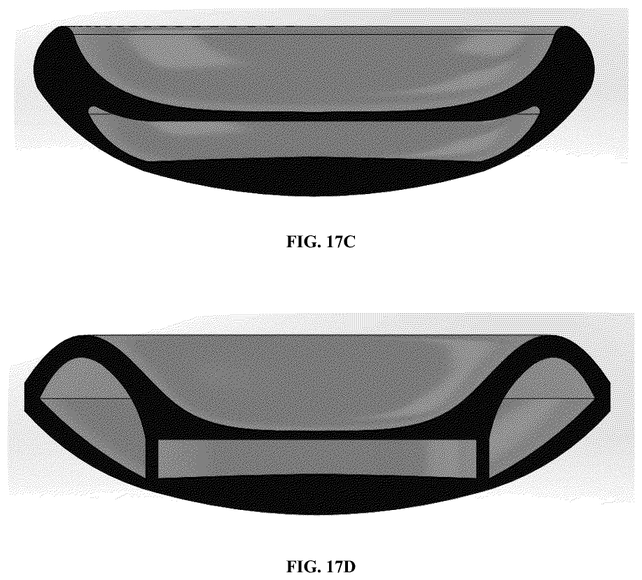

Reference is now made to FIGS. 17A-17D, which illustrate an accommodating intraocular lens with a gas chamber, in this example an air chamber 400, constructed and operative in accordance with an embodiment of the disclosure. In one embodiment (FIG. 17A and FIG. 17B), the air pocket 400 surrounds the membrane, and membrane is found both above and below the air pocket. In another embodiment (FIG. 17C), the air pocket surrounds the membrane is the air pocket extends to the bottom of the lens. The gas pocket is found above, below, right and left of the membrane.

In one embodiment (17D), the air pocket surrounds the membrane and the air pocket extends to the bottom of the lens. Membrane is found to the left and to the right of the air pocket.

The lenses and methods described herein provide diopter changes that are substantially in excess of anything disclosed in the literature. Thus, for example, diopter changes (and accommodation as discussed above) including but not limited to four diopters, six diopters, eight diopters, ten diopters, twelve diopters, or more are obtained in practice of the lenses and methods disclosed herein.

The following patents and published patent applications are incorporated by reference herein: U.S. Patent Application Publication No. 2004/0181279; U.S. Pat. Nos. 7,025,783; and 5,443,506.

The devices, lens, lens assemblies and methods disclosed herein are further described in the following paragraphs:

1. An accommodating intraocular lens as shown in any one of FIGS. 1-20.

2. A method of making refractive power changes of an ophthalmic lens as hereinabove described.

3. An intraocular lens comprising: an optical element adapted to be implanted within the capsular bag of a human eye, wherein the optical element has a posterior lens forming a refractive surface with a positive diopter and a gas chamber adjacent to a polymeric membrane forming a refractive surface of a negative power.

4. An intraocular lens comprising: an optical element adapted to be implanted within a human eye, wherein the optical element has a posterior lens forming a refractive surface with a positive diopter and a gas chamber adjacent to a polymeric membrane forming a refractive surface of a negative power.

5. An intraocular lens comprising: an optical element adapted to be implanted within the capsular bag of a human eye, wherein the optical element includes one a central gas chamber and at least one peripheral gas chamber, wherein the central gas chamber and the peripheral gas chamber each contain a gas with a refractive index of about 1.00.

6. A lens for implantation in the human eye comprising a refractive surface with a positive diopter and a gas chamber containing gas adjacent to a polymeric membrane forming a refractive surface of a negative power.

7. A lens for implantation in the human eye comprising a refractive surface with a positive diopter and a gas chamber containing gas adjacent to a polymeric membrane forming a refractive surface of a negative power, wherein the gas a refractive index of about 1.00.

8. A lens as described in any one of the preceding paragraphs wherein the tension in the ciliary muscle of the eye alters the shape of at least one of the refractive surfaces in such a way that higher tension in the ciliary muscle makes at least one of said refractive surfaces be more concave thereby increasing the positive power of said lens.

9. A lens as described in any one of the preceding paragraphs further comprising haptics to fit in a lens capsule of the eye after the natural lens has been extracted as part of cataract surgery, said haptics are connected in such a manner that compression of said haptics causes reduced pressure in the interior of said lens so as to make at least one of said refractive surfaces of said lens be more concave thereby increasing the positive power of said lens.

10. A lens as described in any one of the preceding paragraphs wherein the refractive surface is resilient.

11. A lens as described in any one of the preceding paragraphs wherein the refractive surface is transparent.

12. A lens assembly having a configuration as shown in FIG. 3A.

13. A lens assembly having a configuration as shown in FIG. 9A.

14. A lens assembly having a configuration as shown in FIG. 11A.

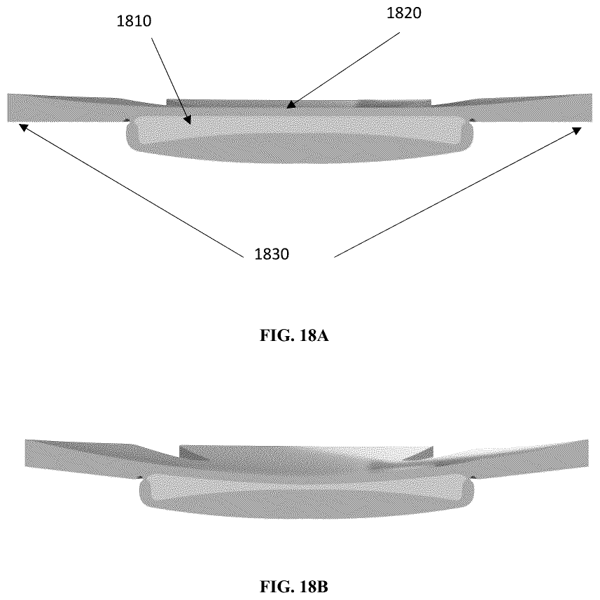

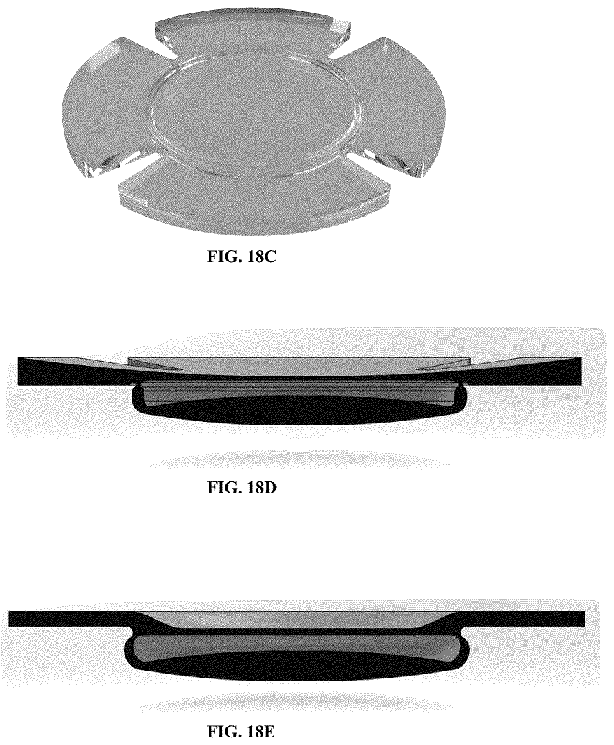

15. A lens assembly having a configuration as shown in FIG. 18A.

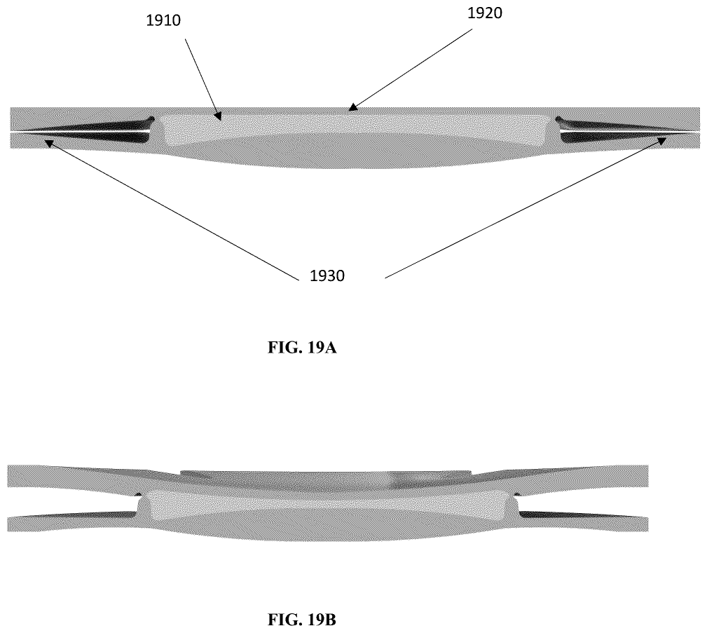

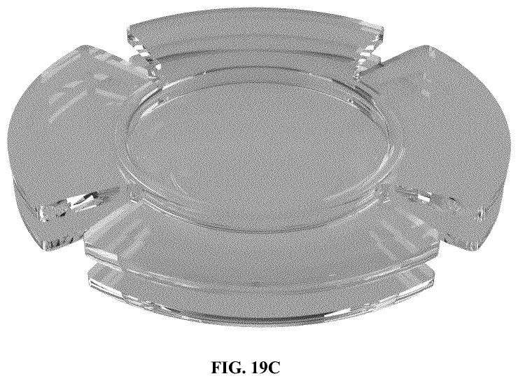

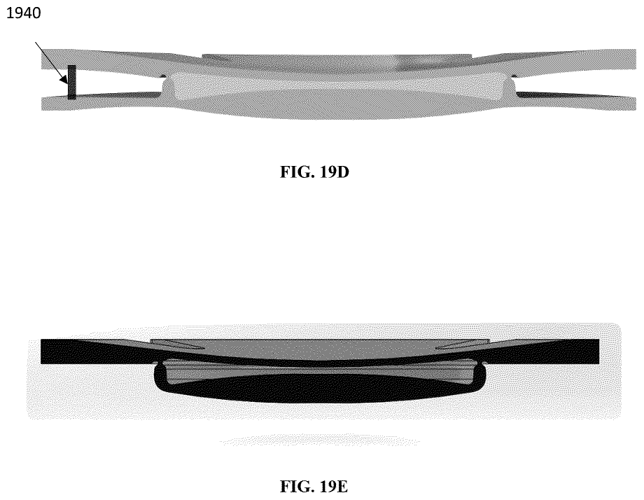

16. A lens assembly having a configuration as shown in FIG. 19A.

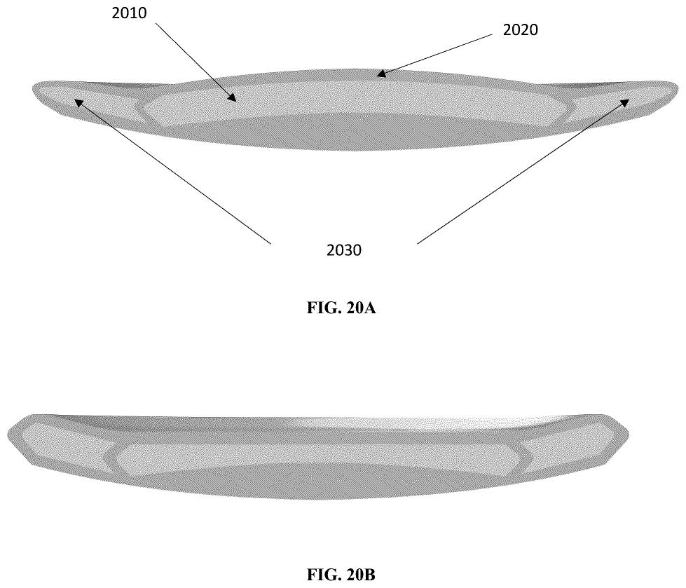

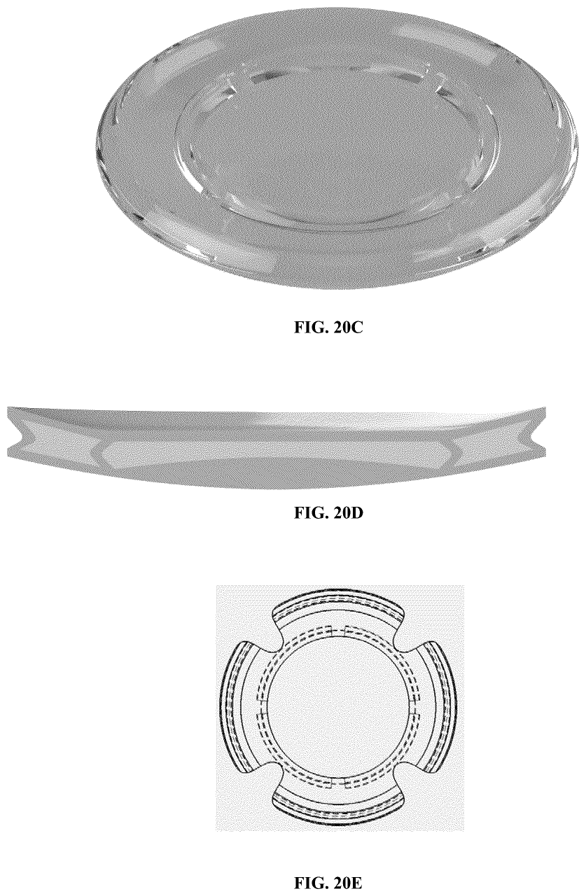

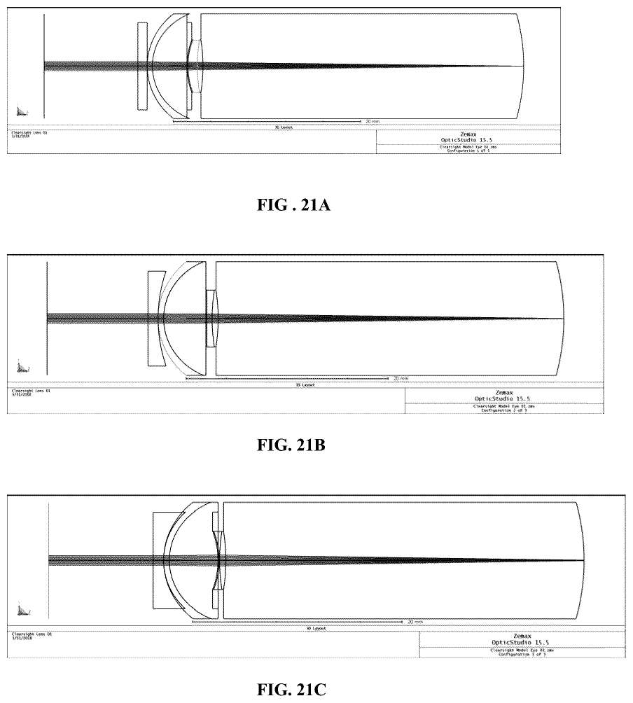

17. A lens assembly having a configuration as shown in FIG. 20A.

18. An intraocular lens comprising: an optical element adapted to be implanted within a human eye, wherein the optical element has a posterior lens forming a refractive surface with a positive diopter and a gas chamber adjacent to a polymeric membrane forming a refractive surface of a negative power, wherein the gas chamber extends to the bottom of the lens.

19. An intraocular lens comprising: an optical element adapted to be implanted within a human eye, wherein the optical element has a posterior lens forming a refractive surface with a positive diopter and a gas chamber adjacent to a polymeric membrane forming a refractive surface of a negative power, wherein the polymer membrane extends to the bottom of the lens.

20. An intraocular lens comprising: a housing having an optical element, wherein the optical element has a posterior lens forming a refractive surface with a positive diopter, and a gas chamber adjacent to a polymeric membrane forming a refractive surface of a negative power, wherein the gas chamber extends to the bottom of the housing.

21. An intraocular lens comprising: a housing having an optical element, wherein the optical element has a posterior lens forming a refractive surface with a positive diopter and a gas chamber adjacent to a polymeric membrane forming a refractive surface of a negative power, wherein the polymer membrane extends to the bottom of the housing.

22. An accommodating intraocular lens for implantation into a generally circular inner surface of an eye, comprising:
(a) a lens body having an optic and having at least two optic hinge portions, a polymeric membrane and a gas chamber containing gas, wherein the gas chamber is adjacent to the polymeric membrane;
(b) at least two haptics, each haptic having a haptic hinge portion pivotally connected to said optic hinge portions, said haptics spaced apart from each other generally radially away from the optic, adapted to engage the generally circular inner surface of the eye for holding the lens in the eye.

23. The accommodating intraocular lens of paragraph 22, wherein each of the haptics includes an outer portion with a surface adapted to engage the generally circular inner surface of the eye, at least part of said outer surface extending beyond the diameter of the generally circular inner surface of the eye, when said outer portion is in its unstressed state, said outer portion being flexible and not conforming to the generally circular inner surface of the eye until subjected to compressive forces, so that the outer surface will conform generally to the shape of the inner surface of the eye when subjected to said compression forces upon implantation.

24. The accommodating intraocular lens of paragraph 23, wherein the haptics being pivotally moveable through a certain angle anteriorly and posteriorly relative to the optic in response to forces imparted to the lens through contraction and expansion of the generally circular inner surface of the eye.

It should be understood that the above described embodiments constitute only examples of an accommodating lens assembly for implantation into the eye according to the disclosure, and that the scope of the methods and lenses disclosed herein fully encompasses other embodiments that may become obvious to those skilled in the art. For example, while implantation of the lens assembly in humans is described, the assembly may clearly also be applicable to other animals. Clearly, any and all possible permutations and/or combinations of different features as described above are within the scope of the present invention.

### EXAMPLES

### Example 1

A model eye was calculated for three IOL configurations depicted in FIGS. 18-20. FIGS. 18A-18E illustrate an accommodating intraocular lens with an air chamber 400, constructed and operative in accordance with an embodiment of the disclosure. FIG. 18A depicts a dis-accommodated/molded state with an air pocket 1810 surrounding the membrane 1820 and hepatics 1830. FIG. 18B depicts an accommodated state with the capsular equator diameter reduction/slope change of the bag causes concave deformation of the membrane, resulting in accommodation. FIG. 18C is an isometric view of the IOL. FIG. 18D displays the variable thickness of the membrane. FIG. 18E displays the offset haptics.

FIG. 19A-19E illustrate an accommodating intraocular lens with an air chamber 1910, constructed and operative in accordance with an embodiment of the disclosure. FIG. 19A depicts a dis-accommodated state with the capsular bag tension causing peripheral haptics 1930 to move together axially, flexing the membrane 1920 to a flat state, resulting in dis-accommodation. FIG. 19B depicts the accommodated state with a concave membrane and the haptics straddling the capsular equator. FIG. 19C is an isometric view of the IOL. FIG. 19D displays the hinged/connected haptics. FIG. 19E depicts the design with the posterior haptics removed.

FIG. 20-20E illustrate an accommodating intraocular lens with an air chamber 2010, constructed and operative in accordance with an embodiment of the disclosure. FIG. 20A depicts a configuration with a central air pocket 2010 as well as a peripheral air pocket 2030. The capsular bag tension causes peripheral air pocket to compress, displacing air from periphery to the central air pocket via channels, bulging central membrane and resulting in dis-accommodation. FIG. 20B depicts the accommodated state where relaxation of the capsular bag tension causes air to return to the lens periphery, causing central membrane to return to a flat state, resulting in accommodation. FIG. 20C is an isometric view of the IOL. FIG. 20D displays the varied hinge mechanism on the periphery. FIG. 20E depicts the design with a haptic cut-out for aqueous flow.

The corneal parameters are standard schematic eye values. The anterior chamber depth for IOL configuration #1 was set to 3.7 mm. For the other two model eye configurations, the IOL posterior lens position was considered to remain stationary and so the model eye anterior chamber depth was adjusted to account for the different IOL axial thicknesses.

The initial axial length of the model eye was determined by first setting up the cornea and the IOL (configuration #1) and then running an optimization to achieve an axial length that provided an emmetropic model eye. The actual optimization used in Zemax was to minimize the image spot size on the retina.

To calculate the refraction of the model eye, a spectacle lens is placed in front of the model eye and an optimization is performed for each IOL configuration to automatically adjust only the posterior spectacle lens surface radius of curvature to achieve an emmetropic eye (row 26 below highlighted in green). Once that optimization is completed, the refractive power of the posterior spectacle lens surface is calculated to determine the refraction of the model eye.

The optimization and refraction calculations are performed for a range of pupil diameters from 0.5 to 6.0 in 0.5 mm steps). As can be seen, the refraction of the model eye changes for different pupil diameters. This is because of the uncorrected spherical aberration of the model eye due to using spherical surfaces. In addition to calculating the refraction, the object distance is also calculated in units of meters, which is the distance an object would have to be positioned relative to the eye to get an in-focus image on the retina (without the spectacle lens in front of the eye).

A negative number (for a myopic eye) means a real object is positioned that distance in front of the eye. A positive number (for a hyperopic eye) means a virtual object has to be positioned that distance behind the eye. In actual fact, this method of calculating the refraction really only applies for paraxial optics (rays very close to the optical axis), so it is really only accurate for small pupil diameters where paraxial optics applies.

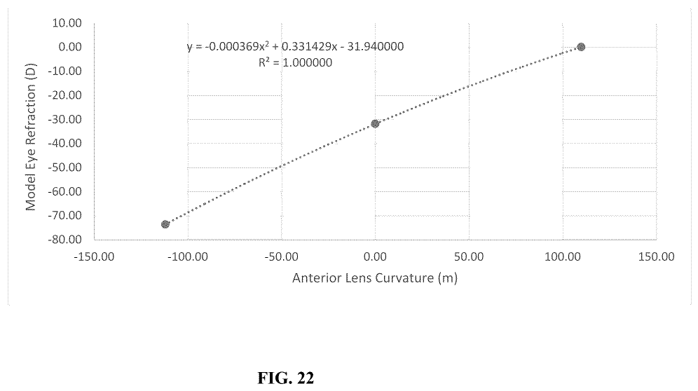

Finally, FIG. 22 plots the model eye refraction against the anterior lens curvature. Here, the anterior IOL surface radius of curvature is converted to curvature in units of meters. Curvature is calculated as 1/radius in units of meters.

TABLE II
Model Eye Parameters for IOL Configuration in FIG. 18A
Model Eye Parameters

| Surface | Radius | Thickness | Index |
|---|---|---|---|
| Air | Infinity | 10.000 | |
| Anterior Spectacle | Infinity | 1.000 | BK7 |
| Posterior Spectacle | 18.276 | 0.000 | |
| Anterior Cornea | 7.800 | 0.550 | 1.362 |
| Posterior Cornea | 6.500 | 3.700 | 1.336 |
| Iris | Infinity | 0.000 | 1.336 |
| Anterior Lens 01 | 9.100 | 0.100 | FIT_LENS |
| Anterior Lens 02 | 9.009 | 1.000 | 1.000 |
| Posterior Lens 01 | 20.120 | 0.600 | FIT_LENS |
| Posterior Lens 02 | −20.120 | 34.212 | 1.336 |

TABLE III
Refraction of Model eye as a function of pupil
diameter for IOL in FIG. 18A
Refraction of Model eye as a function of pupil diameter

| Aperture(mm) | Radius(m) | Refraction(D) | Obj-dist(m) |
|---|---|---|---|
| 0.5000 | −10.8954 | 0.0476 | 21.0124 |
| 1.0000 | −18.0540 | 0.0287 | 34.8183 |
| 1.5000 | 178.3079 | −0.0029 | −343.8769 |
| 2.0000 | 10.9007 | −0.0476 | −21.0226 |
| 2.5000 | 4.9079 | −0.1056 | −9.4652 |
| 3.0000 | 2.9184 | −0.1777 | −5.6283 |
| 3.5000 | 1.9618 | −0.2643 | −3.7835 |
| 4.0000 | 1.4153 | −0.3664 | −2.7294 |
| 4.5000 | 1.0693 | −0.4849 | −2.0623 |
| 5.0000 | 0.8348 | −0.6211 | −1.6100 |
| 5.5000 | 0.6677 | −0.7765 | −1.2878 |
| 6.0000 | 0.5441 | −0.9530 | −1.0493 |

TABLE IV
Model Eye Parameters for IOL Configuration in FIG. 19A
Model Eye Parameters

| Surface | Radius | Thickness | Index |
|---|---|---|---|
| Air | Infinity | 10.0000 | |
| Anterior Spectacle | Infinity | 1.0000 | BK7 |
| Posterior Spectacle | 16.2343 | 0.0000 | |
| Anterior Cornea | 7.8000 | 0.5500 | 1.3620 |
| Posterior Cornea | 6.5000 | 4.1800 | 1.3360 |
| Iris | Infinity | 0.0000 | 1.3360 |
| Anterior Lens 01 | Infinity | 0.1000 | FIT_LENS |
| Anterior Lens 02 | Infinity | 0.5200 | 1.0000 |
| Posterior Lens 01 | 20.1200 | 0.6000 | FIT_LENS |
| Posterior Lens 02 | −20.1200 | 34.2116 | 1.3360 |
| Retina | −24.0000 | | |
| | | 40.1616 | |

TABLE V
Refraction of Model Eye as a Function of Pupil
Diameter for IOL Configuration in FIG. 19A
Refraction of Model eye as a function of pupil diameter

| Aperture(mm) | Radius(m) | Refraction(D) | Obj-dist(m) |
|---|---|---|---|
| 0.5000 | 0.0163 | −31.8947 | −0.0314 |
| 1.0000 | 0.0162 | −31.9400 | −0.0313 |
| 1.5000 | 0.0162 | −32.0159 | −0.0312 |
| 2.0000 | 0.0161 | −32.1230 | −0.0311 |
| 2.5000 | 0.0161 | −32.2622 | −0.0310 |
| 3.0000 | 0.0160 | −32.4346 | −0.0308 |
| 3.5000 | 0.0159 | −32.6417 | −0.0306 |
| 4.0000 | 0.0158 | −32.8854 | −0.0304 |
| 4.5000 | 0.0156 | −33.1678 | −0.0301 |
| 5.0000 | 0.0155 | −33.4919 | −0.0299 |
| 5.5000 | 0.0153 | −33.8611 | −0.0295 |
| 6.0000 | 0.0151 | −34.2796 | −0.0292 |

TABLE VI
Model Eye Parameters for IOL Configuration in FIG. 20A
Model Eye Parameters

| Surface | Radius | Thickness | Index |
|---|---|---|---|
| Air | Infinity | 10.0000 | |
| Anterior Spectacle | Infinity | 1.0000 | BK7 |
| Posterior Spectacle | 7.0370 | 0.0000 | |
| Anterior Cornea | 7.8000 | 0.5500 | 1.3620 |
| Posterior Cornea | 6.5000 | 4.6500 | 1.3360 |
| Iris | Infinity | 0.0000 | 1.3360 |
| Anterior Lens 01 | −8.9286 | 0.1000 | FIT_LENS |
| Anterior Lens 02 | −9.0090 | 0.0500 | 1.0000 |
| Posterior Lens 01 | 20.1200 | 0.6000 | FIT_LENS |
| Posterior Lens 02 | −20.1200 | 34.2116 | 1.3360 |
| Retina | −24.0000 | | |
| | | 40.1616 | |

TABLE VII
Refraction of Model Eye for IOL Configuration in FIG. 20A
Refraction of Model eye as a function of pupil diameter

| Aperture(mm) | Radius(m) | Refraction(D) | Obj-dist(m) |
|---|---|---|---|
| 0.5000 | 0.0070 | −73.7026 | −0.0136 |
| 1.0000 | 0.0070 | −73.6847 | −0.0136 |
| 1.5000 | 0.0070 | −73.6542 | −0.0136 |
| 2.0000 | 0.0070 | −73.6103 | −0.0136 |
| 2.5000 | 0.0070 | −73.5515 | −0.0136 |
| 3.0000 | 0.0071 | −73.4760 | −0.0136 |
| 3.5000 | 0.0071 | −73.3819 | −0.0136 |
| 4.0000 | 0.0071 | −73.2663 | −0.0136 |
| 4.5000 | 0.0071 | −73.1262 | −0.0137 |
| 5.0000 | 0.0071 | −72.9581 | −0.0137 |
| 5.5000 | 0.0071 | −72.7579 | −0.0137 |
| 6.0000 | 0.0071 | −72.5209 | −0.0138 |

TABLE VIII
Model Eye Refraction Calculations

| | | | |
|---|---|---|---|
| Membrane deflection (mm) | −0.1 | | |
| ½ support distance (mm) | 3 | | |
| Radius of curvature of membrane (mm) | −45.05 | Radius in meters | −0.04505 |
| Calculated model eye refraction (D) | −39.48 | 1/Radius (m) | −22.19755827 |
| Calculated accommodation (vs. flat membrane) (D) | 7.54 | | |

### Example 2

Various design configurations were tested and are shown in Table IX. Parameters tested include accommodation range, required force, overall lens thickness, inflation, use of membranes, general manufacturability, complexity of mechanics, injection/foldability properties, aqueous flow, open bag/surface area contact, ease of implementation, and optic system stability. The results are summarized in Table IX.

TABLE IX
Design Evaluation Matrix for Various Configurations
Design Evaluation Matrix

| | | A | B | C | D | E | F |
|---|---|---|---|---|---|---|---|
| Design | | FIG. | FIG. | FIG. | FIG. | FIG. | FIG. |
| Criteria | Weight | 16A | 12A | 5A | 9A | 14A | 6A |
| Accommodation | 3 | 0 | 0 | 0 | 0 | + | + |
| range | | | | | | | |
| Required force | 3 • | 0 | 0 | 0 | 0 | + | + |
| (estimation) | | | | | | | |
| Overall lens | 3 | + | + | 0 | 0 | − | − |
| thickness (incision | | | | | | | |
| size) | | | | | | | |
| Inflation | 1 | + | + | + | + | 0 | 0 |
| (manufacturability) | | | | | | | |
| Use of membranes | 2 | 0 | 0 | 0 | 0 | − | − |
| (manufacturability) | | | | | | | |
| General | 3 | − | − | 0 | 0 | − | − |
| manufacturability | | | | | | | |
| Complexity of | 2 | + | 0 | 0 | 0 | + | + |
| mechanics | | | | | | | |
| Injection/foldability | 3 | + | + | 0 | 0 | − | − |
| properties | | | | | | | |
| Aqueous flow | 2 | + | + | + | + | 0 | 0 |
| Open bag (surface | 3 | + | + | + | 0 | + | 0 |
| area contact) | | | | | | | |
| Ease of | 3 | 0 | 0 | − | − | 0 | − |
| implantation | | | | | | | |
| (surgical technique) | | | | | | | |
| Optic system | 2 | + | + | 0 | + | + | 0 |
| stability | | | | | | | |

What is claimed is:

1. An intraocular lens comprising: an optical element adapted to be implanted within a human eye, wherein the optical element has a posterior lens forming a refractive surface with a positive diopter and a gas chamber containing gas adjacent to a polymeric membrane forming a refractive surface of a negative power.

2. The intraocular lens of claim 1, wherein the gas a refractive index of about 1.00.

3. The intraocular lens of claim 1, wherein the gas is air.

4. A lens for implantation in the human eye comprising a refractive surface with a positive diopter and a gas chamber containing gas adjacent to a polymeric membrane forming a refractive surface of a negative power.

5. The intraocular lens of claim 4, wherein the gas a refractive index of about 1.00.

6. The intraocular lens of claim 4, wherein the gas is air.

7. An accommodating intraocular lens assembly for implantation into a generally circular inner surface of an eye, comprising:
(a) a lens body having an optic and having at least two optic hinge portions, a polymeric membrane and a gas chamber containing gas, wherein the gas chamber is adjacent to the polymeric membrane;
(b) at least two haptics, each haptic having a haptic hinge portion pivotally connected to said optic hinge portions, said haptics spaced apart from each other generally radially away from the optic, adapted to engage the generally circular inner surface of the eye for holding the lens in the eye.

8. The accommodating intraocular lens assembly of claim 7, wherein the optic is a posterior lens.

9. The accommodating intraocular lens assembly of claim 8, wherein the posterior lens forms a refractive surface with a positive diopter.

10. The accommodating intraocular lens assembly of claim 7, wherein polymeric membrane forms a refractive surface of a negative power.

11. The accommodating intraocular lens assembly of claim 7 wherein the gas has a refractive index of about 1.00.

12. The accommodating intraocular lens assembly of claim 7, wherein the gas is air.
